SEQUENCE LISTING (98 sequences)

SEQ ID NO: 1 - DNA, 52 bp, artificial
  primer
tactatggatccaggaggtaacagctatgagtaaaatcgttgtagtcggtgc

SEQ ID NO: 2 - DNA, 50 bp, artificial
  primer
atatagtgatcgatagcagtctgcagttatttttcagccgtaagggcagc

SEQ ID NO: 3 - DNA, 18 bp, Escherichia coli
tacacattcgttaagtca

SEQ ID NO: 4 - DNA, 18 bp, Escherichia coli
tcatttaacggtatgttg

SEQ ID NO: 5 - DNA, 18 bp, Escherichia coli
caatttatccttaaacat

SEQ ID NO: 6 - DNA, 18 bp, Escherichia coli
aactttaaggaaacacaa

SEQ ID NO: 7 - DNA, 18 bp, Escherichia coli
cctttgtttgttaattat

SEQ ID NO: 8 - DNA, 18 bp, Escherichia coli
tggcttatcgaatttccc

SEQ ID NO: 9 - DNA, 18 bp, Escherichia coli
cgtcggtttattaataag

SEQ ID NO: 10 - DNA, 18 bp, Escherichia coli
aacagattcgttcagcaa

SEQ ID NO: 11 - DNA, 18 bp, Escherichia coli
gcaatgaacgaactcgaa

SEQ ID NO: 12 - DNA, 18 bp, Escherichia coli
tgaactaccgaatcagct

SEQ ID NO: 13 - DNA, 18 bp, Escherichia coli
gagtttaacgaagtaatt

SEQ ID NO: 14 - DNA, 18 bp, Escherichia coli
ttggttaacaacctggct

SEQ ID NO: 15 - DNA, 18 bp, Escherichia coli
tagcgttttgttattcga

SEQ ID NO: 16 - DNA, 18 bp, Escherichia coli
ccggttaccgcttctacg

SEQ ID NO: 17 - DNA, 18 bp, Escherichia coli
gttgtacttgtttagcga

SEQ ID NO: 18 - DNA, 18 bp, Escherichia coli
cccggtattgttatttat

SEQ ID NO: 19 - DNA, 16 bp, Escherichia coli
tcgattcgtaaaagtg

SEQ ID NO: 20 - DNA, 18 bp, Escherichia coli
tgaactgttgttcaacat

SEQ ID NO: 21 - DNA, 18 bp, Escherichia coli
atgattaacgctacttat

SEQ ID NO: 22 - DNA, 18 bp, Escherichia coli
tcaggaaccggtaaacgg

SEQ ID NO: 23 - DNA, 18 bp, Escherichia coli
ttttctatcgttaatatt

SEQ ID NO: 24 - DNA, 18 bp, Escherichia coli
taaacataagttaactgg

SEQ ID NO: 25 - DNA, 18 bp, Escherichia coli
attggttttgtaaacctg

SEQ ID NO: 26 - DNA, 18 bp, Escherichia coli
tcacttaaccattccatg

SEQ ID NO: 27 - DNA, 18 bp, Escherichia coli
acgtttatgaaatcactt

SEQ ID NO: 28 - DNA, 18 bp, Escherichia coli
aagatgaaccatgacgtc

SEQ ID NO: 29 - DNA, 18 bp, Escherichia coli
ttaataaacaaaaggtta

SEQ ID NO: 30 - DNA, 18 bp, Escherichia coli
ttaattaaaaataaatca

SEQ ID NO: 31 - DNA, 16 bp, Escherichia coli
ggttaacaccccatgc

SEQ ID NO: 32 - DNA, 18 bp, Escherichia coli
gtggcctgcgttaatgca

SEQ ID NO: 33 - DNA, 18 bp, Escherichia coli
gctttaaacaaacaatca

SEQ ID NO: 34 - DNA, 18 bp, Escherichia coli
gtattaaacaaatgtata

SEQ ID NO: 35 - DNA, 18 bp, Escherichia coli
ttttttgccaaaacgcac

SEQ ID NO: 36 - DNA, 18 bp, Escherichia coli
acctggatcgtgaaaagc

SEQ ID NO: 37 - DNA, 18 bp, Escherichia coli
gcaggctttgtgaaagcc

SEQ ID NO: 38 - DNA, 18 bp, Escherichia coli
caggttattgctgaactg

SEQ ID NO: 39 - DNA, 18 bp, Escherichia coli
ggaatttacaaacttcag

SEQ ID NO: 40 - DNA, 18 bp, Escherichia coli
aactcatttgttaatttt

SEQ ID NO: 41 - DNA, 18 bp, Escherichia coli
gaccgtattgtgaaacgt

SEQ ID NO: 42 - DNA, 18 bp, Escherichia coli
ttatggttggttatgaaa

SEQ ID NO: 43 - DNA, 18 bp, Escherichia coli
gcggaatctgttaataaa

SEQ ID NO: 44 - DNA, 18 bp, Escherichia coli
tgcgttaacgcatcccga

SEQ ID NO: 45 - DNA, 18 bp, Escherichia coli
ctcgttaacgataagttt

SEQ ID NO: 46 - DNA, 15 bp, Escherichia coli
ggtttcgcttacgtt

SEQ ID NO: 47 - DNA, 18 bp, Escherichia coli
gaaattagcgaaaaagta

SEQ ID NO: 48 - DNA, 18 bp, Escherichia coli
ttttgtctcgctaagtta

SEQ ID NO: 49 - DNA, 18 bp, Escherichia coli
gatgttaacgctaaagag

SEQ ID NO: 50 - DNA, 18 bp, Escherichia coli
atcattaacaaaaaattg

SEQ ID NO: 51 - DNA, 18 bp, Escherichia coli
tgttttatttttaattga

SEQ ID NO: 52 - DNA, 18 bp, Escherichia coli
gatttcaaccatccgctg

SEQ ID NO: 53 - DNA, 18 bp, Escherichia coli
cggtgttttgctcatgct

SEQ ID NO: 54 - DNA, 18 bp, Escherichia coli
agaatttttatgaattac

SEQ ID NO: 55 - DNA, 18 bp, Escherichia coli
agggttcacatatattta

SEQ ID NO: 56 - DNA, 18 bp, Escherichia coli
ctgtttaactgatacggt

SEQ ID NO: 57 - DNA, 18 bp, Escherichia coli
aagaccatcgttaacggt

SEQ ID NO: 58 - DNA, 18 bp, Escherichia coli
tcttttagtgttaaattc

SEQ ID NO: 59 - DNA, 18 bp, Escherichia coli
ctacttcacaaatcaaac

SEQ ID NO: 60 - DNA, 18 bp, Escherichia coli
aacagtatcgctaatcgt

SEQ ID NO: 61 - DNA, 18 bp, Escherichia coli
tgagaattcgttgaaaat

SEQ ID NO: 62 - DNA, 18 bp, Escherichia coli
gtcagttttgtttacgat

SEQ ID NO: 63 - DNA, 18 bp, Escherichia coli
agcattaacattatctta

SEQ ID NO: 64 - DNA, 18 bp, Escherichia coli
gccgttaaaaatttattc

SEQ ID NO: 65 - DNA, 18 bp, Escherichia coli
gagtttttcaataaccgc

SEQ ID NO: 66 - DNA, 18 bp, Escherichia coli
aacattaatgaacagtct

SEQ ID NO: 67 - DNA, 18 bp, Escherichia coli
ggtattaaaaatgagatt

SEQ ID NO: 68 - DNA, 18 bp, Escherichia coli
cagcaaattgtgaacatc

SEQ ID NO: 69 - DNA, 18 bp, Escherichia coli
cgccgaagcgtcaacatg

SEQ ID NO: 70 - DNA, 18 bp, Escherichia coli
aatgattttgtgaacagc

SEQ ID NO: 71 - DNA, 18 bp, Escherichia coli
ttaattaactataatgaa

SEQ ID NO: 72 - DNA, 18 bp, Escherichia coli
gagagttttgctgaactc

SEQ ID NO: 73 - DNA, 18 bp, Escherichia coli
gccgttctggttaacatc

SEQ ID NO: 74 - DNA, 18 bp, Escherichia coli
ttgtgatttgttgattta

SEQ ID NO: 75 - DNA, 18 bp, Escherichia coli
atctttaccaattttatt

SEQ ID NO: 76 - DNA, 18 bp, Escherichia coli
cgcgtaaacaaactggaa

SEQ ID NO: 77 - DNA, 18 bp, Escherichia coli
ggctgaatcgttaaggat

SEQ ID NO: 78 - DNA, 16 bp, Escherichia coli
accgttagcgagtaat

SEQ ID NO: 79 - DNA, 18 bp, Escherichia coli
acacttaactgtacaagt

SEQ ID NO: 80 - DNA, 18 bp, Escherichia coli
cgtcattttgtgaatgca

SEQ ID NO: 81 - DNA, 18 bp, Escherichia coli
acctttaaaaaataacca

SEQ ID NO: 82 - DNA, 18 bp, Escherichia coli
acattcaacaaggaaaga

SEQ ID NO: 83 - DNA, 18 bp, Escherichia coli
tatttttacaattcacat

SEQ ID NO: 84 - DNA, 18 bp, Escherichia coli
attttgaacaatatggca

SEQ ID NO: 85 - DNA, 18 bp, Escherichia coli
tcagttttccttcatcat

SEQ ID NO: 86 - DNA, 18 bp, Escherichia coli
ctggttacccattcctta

SEQ ID NO: 87 - DNA, 18 bp, Escherichia coli
ggattttacaaaaagctc

SEQ ID NO: 88 - DNA, 18 bp, Escherichia coli
ggttttatcaaaccgcgc

SEQ ID NO: 89 - DNA, 18 bp, Escherichia coli
cacattttcgttaatcaa

SEQ ID NO: 90 - DNA, 18 bp, Escherichia coli
gtttttaacaattccgta

SEQ ID NO: 91 - DNA, 18 bp, Escherichia coli
acccataacgataaccgg

SEQ ID NO: 92 - DNA, 18 bp, Escherichia coli
atcttttttgtcactttt

SEQ ID NO: 93 - DNA, 18 bp, Escherichia coli
ttcattcacaatactgga

SEQ ID NO: 94 - DNA, 18 bp, Escherichia coli
tctgttaacaaacgcggt

SEQ ID NO: 95 - DNA, 18 bp, Escherichia coli
acagttaacgactatcgc

SEQ ID NO: 96 - DNA, 18 bp, Escherichia coli
agcgttaaaaaatgagtg

SEQ ID NO: 97 - DNA, 18 bp, Escherichia coli
ttgttgttcgttatggtc

SEQ ID NO: 98 - DNA, 15 bp, Escherichia coli
gtwgryvdgakmwsv

(19) United States
(12) Patent Application Publication
(10) Pub. No.: US 2007/0249018 A1
(43) Pub. Date: Oct. 25, 2007

(54) Reduced overflow metabolism and methods of use

(76) Inventors: Goutham Vemuri, Lyngby (DK); Mark A. Eiteman, Athens, GA (US); Elliot Altman, Athens, GA (US)

Correspondence Address: MUETING, RAASCH & GEBHARDT, P.A., P.O. BOX 581415, MINNEAPOLIS, MN, 55458, US

(21) Appl. No.: 11/710,333

(22) Filed: Feb. 23, 2007

Related U.S. Application Data
(60) Provisional application No. 60/776,032, filed on Feb. 23, 2006.

(51) Int. Cl. C12P 1/04; C12N 1/21

(52) U.S. Cl. 435/69.1; 435/243; 435/252.8

(57) ABSTRACT

The present invention provides modified bacterial cells and methods for using them. A modified bacterial cell can exhibit increased NADH oxidase activity, decreased ArcA activity, or the combination thereof. The methods include culturing a modified bacterial cell in aerobic conditions. The modified bacterial cell can produce less acetate during the culturing than the unmodified bacterial cell under comparable conditions. In some aspects, the modified bacterial cell produces a recombinant polypeptide, and the bacterial cell may produce more recombinant polypeptide than the unmodified bacterial cell under comparable conditions.

### CONTINUING APPLICATION DATA

This application claims the benefit of U.S. Provisional Application Ser. No. 60/776,032, filed Feb. 23, 2006, which is incorporated by reference herein.

### GOVERNMENT FUNDING

The present invention was made with government support under Grant No. DE-FG36-01ID14007, awarded by the U.S. Department of Energy, and Grant No. QSB 0222636, awarded by the National Science Foundation. The Government has certain rights in this invention.

### BACKGROUND

Acetate formation in aerobically grown cultures of Escherichia coli continues to be a major problem in the industrial application of this organism. E. coli accumulates acetic acid when growing at a high rate of glucose consumption even in the presence of ample oxygen (Andersen et al., 1980, J. Bacteriol. 144:114-123, Hollywood et al., 1976, Microbios. 17:23-33; Meyer et al., 1984, J. Biotechnol. 1:355-358). This phenomenon is known as overflow metabolism. Acetate is generated when carbon flux from acetyl-coenzyme A (CoA) is directed to acetate instead of entering the tricarboxylic acid (TCA) cycle (Hollywood et al., 1976, Microbios. 17:23-33). This by-product induces a stress response even at extremely low concentrations (Kirkpatrick et al., 2001, J. Bacteriol. 183:6466-6477), hinders growth (Luli et al., 1990, Appl. Environ. Microbiol. 56:1004-1011), and reduces the production of recombinant proteins (Swartz, 2001. Curr. Opin. Biotechnol. 12:195-201). Overflow metabolism has been attributed to an enzymatic limitation in the TCA cycle (Majewski et al., 1990, Biotechnol. Bioeng. 35:732-738. In E. coli the complete oxidation of 1 mol of glucose in glycolysis and the TCA cycle generates 10 mol of NAD(P)H and 2 mol of FADH₂ (Neidhardt et al., 1990, Physiology of the Bacterial Cell: a Molecular Approach, Sinauer Associates, Sunderland, Mass.).
Glucose+8NAD⁺+2NADP⁺+2FAD+4ADP+4Pi→6CO₂+8NADH+2NADPH+2FADH₂+4ATP+10H⁺

If the rate of oxygen utilization is sufficiently high, the reduced cofactors generated by glucose consumption are reoxidized in the electron transport chain, which serves the dual purpose of maintaining an optimal redox environment and generating energy by oxidative phosphorylation. In the absence of oxygen glucose cannot be completely oxidized, and metabolic intermediates accumulate to maintain the redox balance. Even in the presence of oxygen, if the rate of glucose consumption is greater than the capacity to reoxidize the reduced equivalents generated, the response is similar to what is observed under anaerobic conditions (Andersen et al., 1977, J. Biol. Chem. 252:4151-4156, Andersen et al., 1980, J. Bacteriol. 144:114-123, Holms, 2001, Adv. Microb. Physiol. 45:271-340). Since the flux from acetyl-CoA to acetate does not generate any NADH while the flux from acetyl-CoA through the TCA cycle generates 8NAD(P)H and 2FADH₂, carbon flow diversion to acetate could be viewed as a means to reduce or prevent further NAD(P)H accumulation (El-Mansi et al., 1989, J. Gen. Microbiol. 135(11):2875-2883, Holms, 2001, Adv. Microb. Physiol. 45:271-340). These inferences regarding acetate overflow have been based on physiological observations and in vitro enzyme assays, and the genetic trigger has not been identified. Details of pathways involved in acetate generation and consumption, including specific enzymes and their regulation, have recently been reviewed (Wolfe, 2005, Microbiol. Mol. Biol. Rev. 69(1):12-50).

A large portion of the literature on E. coli physiology focuses on eliminating acetate formation by genetic manipulation (San et al., 1994, Ann. N.Y. Acad Sci. 721:257-267, Chou et al., 1994, Biotechnol. Prog. 10(6):644-647, Aristidou et al., 1995, Biotechnol. Prog. 11 (4):475-478) or process control (Konstantinov et al., 1990, Biotechnol. Bioeng. 36:750-758, Kleman et al., 1994, Appl. Environ. Microbiol. 60(11):3952-3958, Riesenberg et al., 1999, Appl. Microbiol. Biotechnol. 51(4):422-430, Akesson et al., 2001, Biotechnol. Bioeng. 73(3):223-230, Johnston et al., 2003, Biotechnol. Bioeng. 84(3)):314-323). Although these strategies reduce acetate formation, they often sacrifice cell growth rate and/or cell performance. Overexpression of anaplerotic enzymes which affect pathways that replenish the TCA cycle, also reduce acetate formation (Farmer, W. and J. C. Liao. 1997, Appl. Environ. Microbiol. 63(8):3205-3210, Gokam et al., 2001, Appl. Microbiol. Biotechnol. 56(1-2):188-195) and increase recombinant protein production (March et al., 2002, Appl. Environ. Microbiol. 68(11):5620-5624).

### SUMMARY OF THE INVENTION

The present invention provides modified modified bacterial cells and methods for using them. A modified bacterial cell may be an obligative aerobe or a facultative aerobe, and can exhibit greater NADH oxidase activity than a wild-type bacterial cell and decreased ArcA activity when compared to the wild-type bacterial cell. The modified bacterial cell may include a heterologous NADH oxidase polypeptide, and the modified bacterial cell may also include an arcA coding region which includes a mutation.

The present invention provides methods including culturing a modified bacterial cell in aerobic conditions. The modified bacterial cell, for instance, an E. coli, exhibits greater conversion of NADH to NAD than a wild-type bacterial cell, greater expression of an aerobic metabolism polypeptide than a wild-type bacterial cell, or both. The modified bacterial cell produces less acetate during the culturing than the unmodified bacterial cell under comparable conditions. The modified bacterial cell may exhibit increased NADH oxidase activity, and the increased NADH oxidase activity may be the result of a heterologous NADH oxidase polypeptide present in the modified bacterial cell. The bacterial cell may exhibit decreased ArcA activity, and the decreased ArcA activity may be the result of an endogenous arcA coding region or arcB coding region present in the modified bacterial cell having a mutation, such as a deletion. The ArcA activity or ArcB activity may be completely eliminated. The modified bacterial cell may produce at least 40% less acetate than the wild-type bacterial cell when cultured under comparable conditions, and the modified bacterial cell may produce a recombinant polypeptide.

The present invention further provides methods including culturing a modified bacterial cell in aerobic conditions and obtaining a desired product produced by the modified bacterial cell. The method may further include isolating the desired product. The modified bacterial cell, for instance, an E. coli, includes increased NADH oxidase activity when compared to a wild-type bacterial cell, decreased ArcA activity when compared to a wild-type bacterial cell, or both. The modified bacterial cell produces more of the desired product than the wild-type bacterial cell under comparable conditions. The desired product may be a metabolite or a recombinant polypeptide, and the modified bacterial cell may produce at least 25% more recombinant polypeptide than the wild-type bacterial cell.

The terms “comprises” and variations thereof do not have a limiting meaning where these terms appear in the description and claims. Unless otherwise specified, “a,” “an,” “the,” and “at least one” are used interchangeably and mean one or more than one.

For any method disclosed herein that includes discrete steps, the steps may be conducted in any feasible order. And, as appropriate, any combination of two or more steps may be conducted simultaneously.

It is to be understood that the terms used herein to describe acids (for example, the term aspartate) are not meant to denote any particular ionization state of the acid, and are meant to include both protonated and unprotonated forms of the compound. Thus, the terms aspartate and aspartic acid refer to the same compound and are used interchangeably.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Steady-state physiological profiles of E. Coli in the presence of heterologous NADH oxidase. Y_{X/S} (⋄, ♦) and q_{A} (◯, ●) values are compared for NOX⁻ (open symbols and dashed lines) and NOX⁺ (solid symbols and lines) as functions of the specific glucose consumption rate. The highest dilution rate studied was about 80% of μₘₐₓ for both strains. The arrows indicate for each strain the critical specific glucose consumption rates at which acetate formation commenced.

FIG. 2. Steady-state respiration for NOX⁻ (open symbols and dashed lines) and NOX⁺ (solid symbols and lines). The steady-state q_{O2} (Δ, ▴) and q_{CO2} (∇, ▾) values are shown as functions of q_{S}.

FIG. 3. In vivo molar concentration ratio of NADH/NAD for NOX⁻ (□) and NOX⁺ (▪) as functions of q_{S}. The critical value of the NADH/NAD ratio at which acetate formation commences is about 0.06 for both NOX⁻ and NOX⁺ (indicated by vertical lines). q_{A} values are also shown for NOX⁻ (◯) and NOX⁺ (●) as functions of q_{S}.

FIG. 4. Intracellular concentrations of key glycolysis metabolites glucose-6-phosphate, fructose-6-phosphate, PEP, pyruvate, and acetyl-CoA were measured under steady-state conditions in NOX⁻ (A) and NOX⁺ (B). q_{A} values are also shown for NOX⁻ (open circles and dashed lines) and NOX⁺ (filled circles and solid lines) as functions of q_{S}.

FIG. 5. Transcriptional profile of central metabolic pathways for NOX⁻ (dashed lines) and NOX⁺ (solid lines). The mean values of the expression ratios are shown for all genes involved in glycolysis, the TCA cycle, the pentose phosphate pathway, and respiration as functions of q_{S}. Vertical lines show the demarcation between respiratory and respirofermentative metabolism for NOX⁻ (dotted) and for NOX⁺ (solid). See FIG. 9 for detailed expression profiles of individual genes.

FIG. 6. (Left) Hierarchical clustering of genes (rows) that are correlated (R>0.9 or R<−0.9) with the redox ratio (NADH/NAD) in NOX⁻ as a function of increasing q_{S} (columns). (Right) Significantly overrepresented functional categories are shown in the table, along with the number of genes in each category and the P value of its significance as calculated using a hypergeometric distribution. Several key genes involved in the TCA cycle, respiration, and biosynthesis exhibited a strong negative correlation with the redox ratio. A large portion of the genes negatively correlated to the redox ratio were partially classified, revealing redox-dependent regulation of many of these genes.

FIG. 7. Physiological characterization of ARCA⁻NOX⁻ (open symbols and dashed lines) and ARCA⁻NOX⁺ (solid symbols and lines) in accelerostat cultures. Y_{X/S} (⋄, ♦) and q_{A} (◯, ●) values are compared as functions of specific glucose consumption rate. The steady-state values of these parameters, obtained for NOX⁻ (dashed lines without symbols) and NOX⁺ (solid lines without symbols) by using chemostats, are also shown.

FIG. 8. Respiration of ARCA⁻NOX⁻ (open symbols and dashed lines) and ARCA⁻NOX⁺ (solid symbols and lines) in accelerostat cultures. q_{O2} (Δ, ▴) and q_{CO2} (∇, ▾) values are compared as functions of specific glucose consumption rate. The steady-state values of these parameters, obtained for NOX⁻ (dashed lines without symbols) and NOX⁺ (solid lines without symbols) by using chemostats, are also shown.

FIG. 9. The relationship between dilution rate and specific glucose consumption rate (q_{S}, g/g DCW h) is shown for NOX⁻ (dashed lines) and NOX⁺ (solid lines). Also, the mean gene expression ratios of the central metabolic coding regions are shown as a function of q_{S}. Mean gene expression ratios of the amino acid metabolism pathways are shown as a function of q_{S} (specific glucose consumption rate). The set of coding regions involved in each pathway (amino acid biosynthesis, nucleotide biosynthesis, transporter family as well as unclassified genes) were obtained from Ecocyc database (Encyclopedia of Escherichia coli K-12 Genes and Metabolism, available on the World Wide Web at ecocyc.org). Expression ratios for NOX⁻ (dashed lines) are shown relative to the gene expression for NOX⁻ at q_{S}=0.22 g/g DCW h, which corresponded to a growth rate of 0.1 h⁻¹, while those for NOX⁺ (solid lines) are relative to gene expression for NOX⁺ at q_{S}=0.20 g/g DCW h, which corresponded to a specific growth rate of 0.06 h⁻¹.

FIG. 10. Growth profiles of the four strains. Glucose (●), biomass (▪) and acetate (▴). Each fermentation was terminated when the glucose had been consumed.

FIG. 11. Specific oxygen uptake rate (A) and specific carbon dioxide evolution rate (B) in E. coli strains NOX⁻ (◯), NOX⁺ (□), ArcA⁻NOX⁻, (●) and ArcA⁻NOX⁺ (▪).

FIG. 12. Production of β-galactosidase in E. coli strains: NOX⁻ (◯), NOX⁺ (□), ArcA⁻NOX⁻, (●) and ArcA⁻NOX⁺ (▪).

### DETAILED DESCRIPTION OF ILLUSTRATIVE EMBODIMENTS

Metabolic engineering includes genetically overexpressing particular enzymes at points in a metabolic pathway, and/or blocking the synthesis of other enzymes, to overcome or circumvent metabolic “bottlenecks.” A goal of metabolic engineering is to optimize the rate and conversion of a substrate into a desired product. The present invention employs a unique metabolic engineering approach which overcomes metabolic limitations that occur when cells produce acetate as an extracellular co-product of aerobic growth. Acetate is undesirable because it retards growth, inhibits polypeptide formation, and represents a diversion of carbon that could otherwise be used to generate biomass and/or a polypeptide product. It has been surprisingly found that cells can be modified to produce less acetate, and in some aspects, no detectable levels of acetate. Advantageously, in certain aspects the bacterial cells of the present invention may also be used to increase production of recombinant polypeptides, increase specific glucose consumption, or increase the production of metabolites.

Bacterial cells useful in the present invention are typically modified. As used herein, a “modified bacterial cell” is a cell that has a different phenotype when compared to the same bacterial cell that differs only with respect to the modification or modifications (also referred to herein as a wild-type cell) grown under comparable conditions. The altered phenotype of a modified bacterial cell is typically due to the presence in the cell of polynucleotides that may or may not encode a polypeptide, the removal of polynucleotides from the cell, or a combination thereof.

In one aspect of the present invention a useful bacterial cell may be modified to include a polypeptide that increases the conversion of NADH present in a cell to NAD, for instance by increasing the amount of NADH converted to NAD and/or increasing the rate at which NADH is converted to NAD. As used herein, the term “polypeptide” refers broadly to a polymer of two or more amino acids joined together by peptide bonds. The term “polypeptide” also includes molecules which contain more than one polypeptide joined by a disulfide bond, or complexes of polypeptides that are joined together, covalently or noncovalently, as multimers (e.g., dimers, tetramers). Thus, the terms enzyme, peptide, oligopeptide, and protein are all included within the definition of polypeptide and these terms are used interchangeably. It should be understood that these terms do not connote a specific length of a polymer of amino acids, nor are they intended to imply or distinguish whether the polypeptide is produced using recombinant techniques, chemical or enzymatic synthesis, or is naturally occurring. A “recombinant polypeptide” refers to a polypeptide produced by a heterologous coding region present in a cell. A recombinant polypeptide is often a polypeptide produced in limited quantities from natural sources, but produced in greater quantities by bacterial cells described herein. An “aerobic metabolism polypeptide” refers to a polypeptide that catalyzes a step in a metabolic pathway that occurs in the presence of O₂. Examples of such metabolic pathways include, for instance, the TCA cycle.

Whether a cell has increased conversion of NADH to NAD can be determined by evaluating the NADH/NAD ratio in a modified bacterial cell and comparing it to the NADH/NAD ratio in a wild-type cell (a bacterial cell identical to the modified cell except for the modification). Methods for determining this ratio are known and used routinely. For instance, a culture of cells, such as a 10 milliliter aliquot, can be rapidly frozen and the cell pellet suspended in either 0.2 M HCl (for extracting NAD) or 0.2 M NaOH (for extracting NADH). The nucleotides can be extracted by boiling the cell suspension and then measured by use of a cycling assay (Bernofsky and Swan, 1973, Anal. Biochem., 53:452-458). The cycling assay involves the transfer of reducing equivalents from NADH ultimately to 3-[4,5-dimethylthiazol-2-yl]-2,5-diphenyltetrazolium bromide (MTT) to measure the specific nucleotides. The rate of reduction of MTT as measured at 570 nm is proportional to the concentration of NADH or NAD (Leonardo et al., J. Bacteriol., 1993, 175:870-878).

An example of a modified bacterial cell having increased conversion of NADH to NAD is a bacterial cell modified to include increased NADH oxidase activity. The term “NADH oxidase” means a molecule that has NADH oxidase activity, i.e., a molecule that catalyzes the oxidization of NADH to generate NAD. The term “NADH oxidase” thus includes, but is not limited to, naturally occurring NADH oxidase enzymes. The increased NADH oxidase activity expressed by the bacterial cell can be due to expression of an endogenous NADH oxidase or a heterologous NADH oxidase. A “heterologous” enzyme is one that is encoded by a coding region that is not normally present in the cell, or a coding region that is normally present in a microbe but is operably linked to a regulatory region to which it is not normally operably linked. For example, a modified bacterial cell that expresses a coding region from a different genus or species that encodes an NADH oxidase contains a heterologous NADH oxidase. Likewise, a modified bacterial cell that expresses a second coding region encoding an NADH oxidase from the same species and operably linked to a different regulatory region also contains a heterologous NADH oxidase. As used herein, the term “polynucleotide” refers to a polymeric form of nucleotides of any length, either ribonucleotides or deoxynucleotides, and includes both double- and single-stranded DNA and RNA. A polynucleotide may include nucleotide sequences having different functions, including for instance coding regions, and non-coding regions such as regulatory sequences. Coding region and regulatory sequence are defined below. A polynucleotide can be obtained directly from a natural source, or can be prepared with the aid of recombinant, enzymatic, or chemical techniques. A polynucleotide can be linear or circular in topology. A polynucleotide can be, for example, a portion of a vector, such as an expression or cloning vector, or a fragment. A “coding region” is a polynucleotide that encodes a polypeptide and, when placed under the control of appropriate regulatory sequences expresses the encoded polypeptide. The boundaries of a coding region are generally determined by a translation start codon at its 5′ end and a translation stop codon at its 3′ end. A regulatory sequence is a polynucleotide that regulates expression of a coding region to which it is operably linked. Nonlimiting examples of regulatory sequences include promoters, transcription initiation sites, translation start sites, translation stop sites, and terminators. “Operably linked” refers to a juxtaposition wherein the components so described are in a relationship permitting them to function in their intended manner. A regulatory sequence is “operably linked” to a coding region when it is joined in such a way that expression of the coding region is achieved under conditions compatible with the regulatory sequence.

Whether a modified bacterial cell has an increased NADH oxidase activity can be determined by measuring the activity of NADH oxidase in an extract of the cell, and comparing the NADH oxidase activity to the NADH oxidase activity present in the wild-type cell (a bacterial cell identical to the modified cell except for the modification). Methods for measuring NADH oxidase activity are known in the art and used routinely. One example is described by Lopez de Felipe et al. (1998, J. Bacteriol. 180(15):3804-3808). Briefly, after rupturing bacterial cells and removing cellular debris, the disappearance of NADH in the presence of EDTA can be measured. For example, cell extract (0.5 to 5 μl) is added to a solution (50 mM potassium phosphate buffer (pH 7.0), 0.29 mM NADH, and 0.3 mM EDTA in a total volume of 1 ml before addition of the cell extract), and the decrease A₃₄₀ is assayed spectrophotometrically at 25° C. A unit of enzyme can be defined as the amount which catalyzed the oxidation of 1 μmol of NADH to NAD per min at 25° C. A bacterial cell is considered to have increased NADH oxidase activity if it has at least 0.1 units, at least 0.2 units, or at least 0.3 units of NADH oxidase/milligram of cell protein when compared to the wild-type cell grown under comparable conditions.

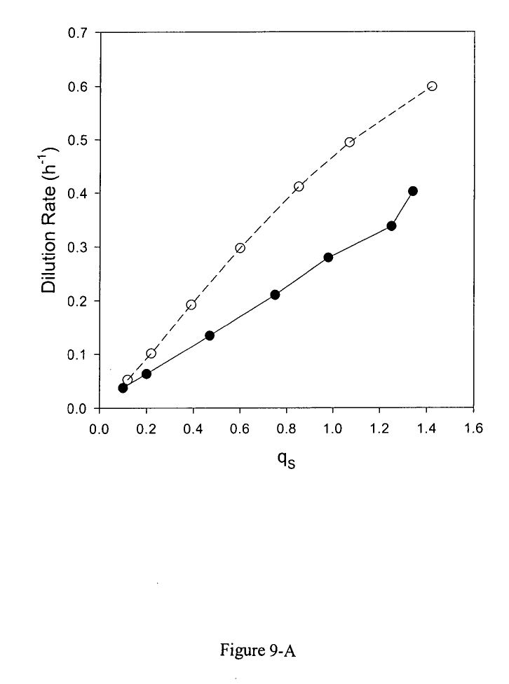

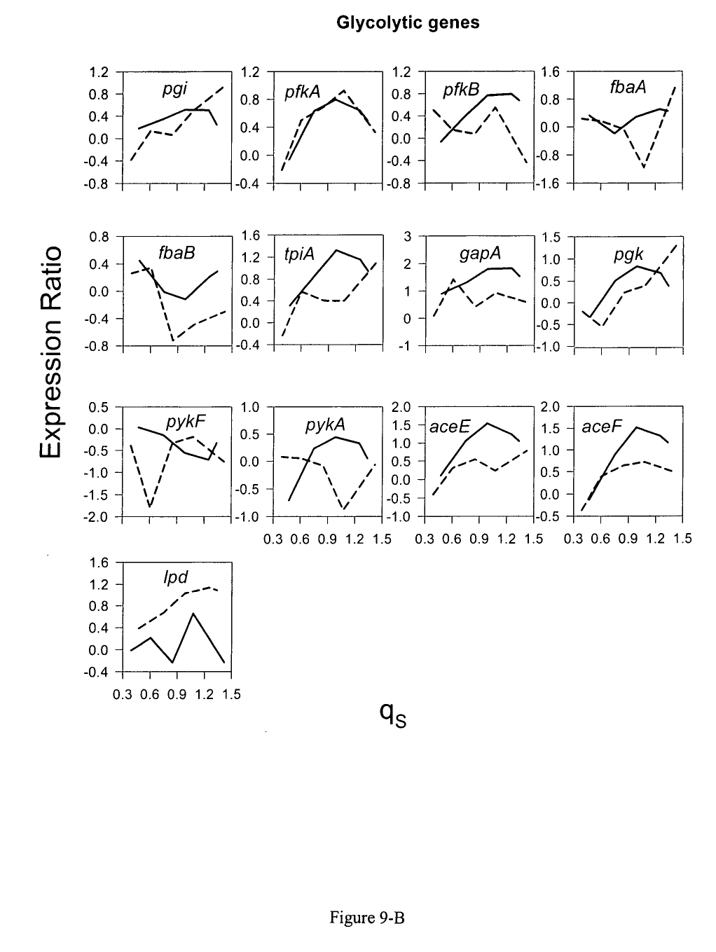

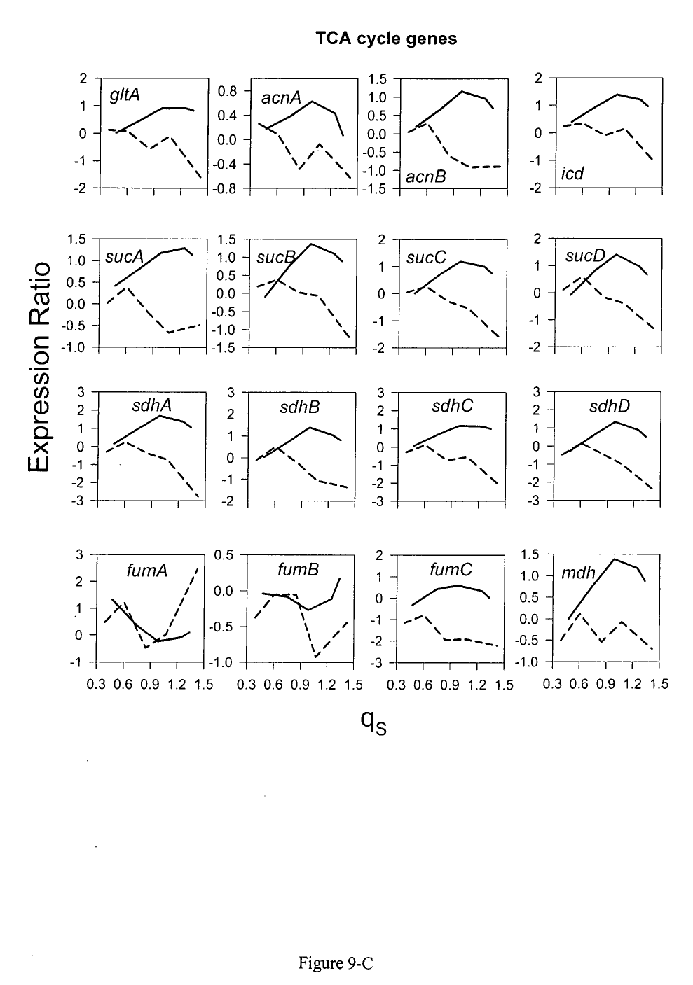

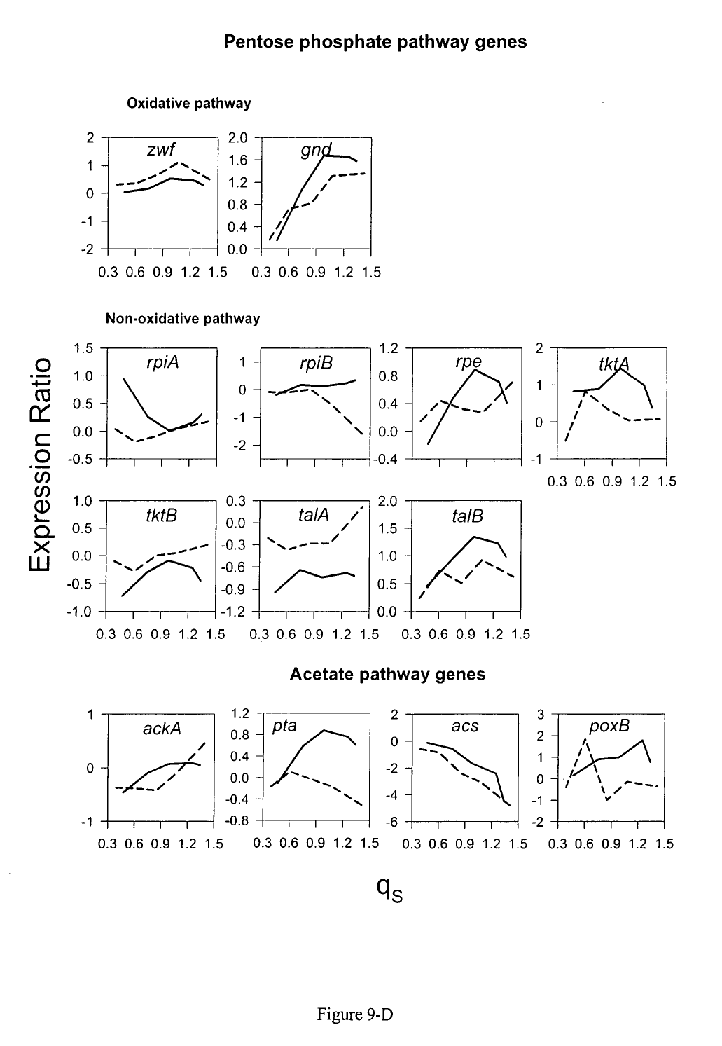

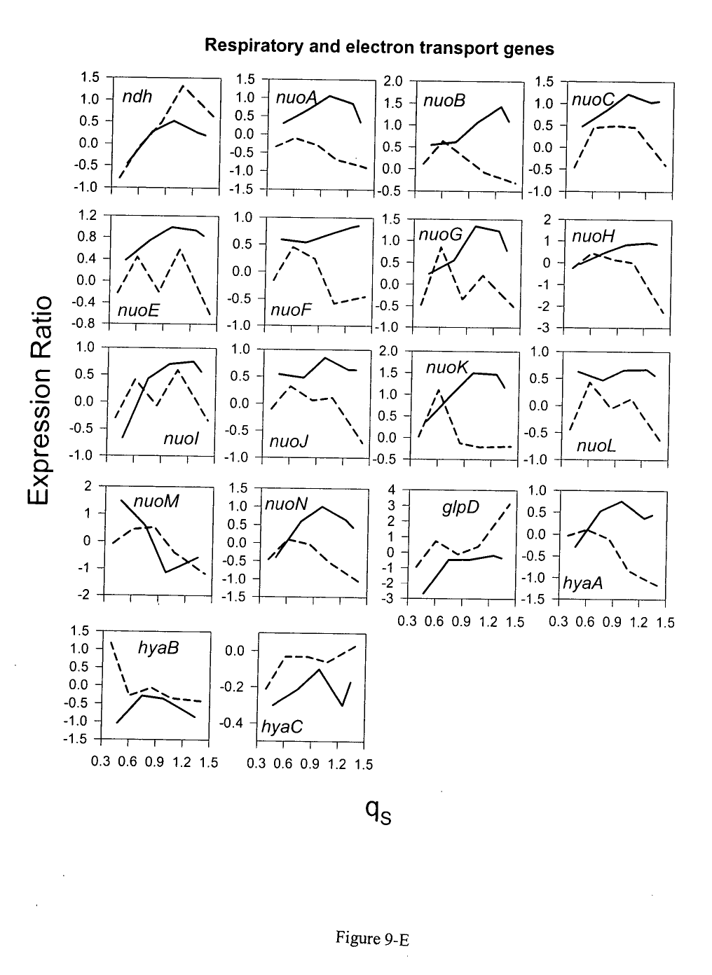

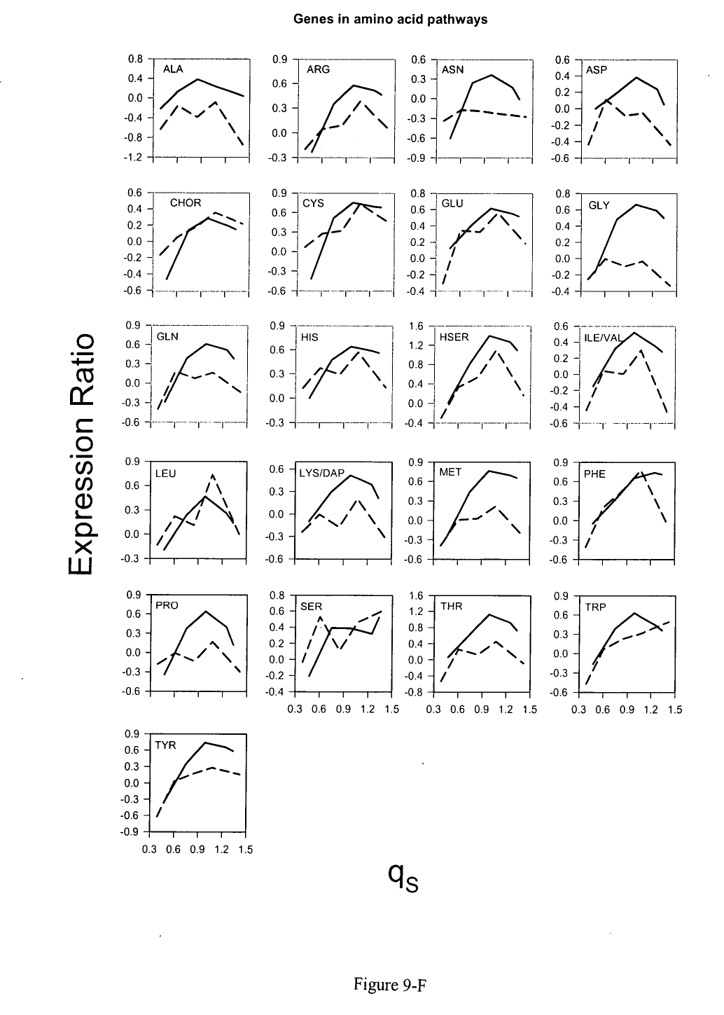

In another aspect of the present invention a useful bacterial cell may be modified to include increased expression of enzymes involved in aerobic metabolism (an aerobic metabolism polypeptide) compared to the wild-type cell (a bacterial cell identical to the modified cell except for the modification). For instance, a bacterial cell can be modified to express polypeptide(s) involved in the TCA cycle and/or respiration, a bacterial cell may be modified to decrease repression of genes encoding polypeptide(s) involved in the TCA cycle and/or respiration, or a combination thereof. Examples of polypeptide(s) involved in aerobic metabolism that can be increased include those encoded by the coding regions disclosed in FIG. 9.

An example of a modified bacterial cell having increased expression of enzymes involved in aerobic metabolism is a bacterial cell modified to have decreased activity of polypeptides involved in the regulation of aerobic metabolism. Examples of such polypeptides are those encoded by the arcA, arcB, fnr, soxR, soxS, oxyR, oxyS, crp, fur, and rpoS coding regions. In one aspect, an example of a modified bacterial cell having increased expression of enzymes involved in the TCA cycle and/or respiration is a bacterial cell modified to have decreased ArcA activity. As used herein, “ArcA” and “arcA” refer to a polypeptide and coding region, respectively. ArcA is one polypeptide of the two-component regulatory ArcAB system, and controls expression of many operons in E. coli and other gram negative bacteria. Some operons controlled by ArcA encode enzymes associated with aerobic metabolism, and ArcA causes decreased expression of many of those operons during anaerobic growth and during aerobic conditions at high growth rate (e.g., exponential growth). Whether a modified bacterial cell has decreased ArcA activity can be determined by measuring the expression of coding sequences controlled by ArcA and comparing their expression with the expression present in a wild-type cell (a bacterial cell identical to the modified cell except for the modification). The modified bacterial cell and the wild-type cell are grown under comparable conditions, and the expression of coding sequences repressed by ArcA are measured. Examples of coding sequences repressed by ArcA during anaerobic growth include, but are not limited to, those encoding the enzymes succinate dehydrogenase, citrate synthase, aconitase, isocitrate dehydrogenase, 2-oxoglutarate dehydrogenase, malate dehydrogenase, fumarase, pyruvate dehydrogenase, isocitrate lyase, acyl-CoA dehydrogenase, 3-hydroxyacyl-CoA dehydrogenase, L-lactate dehydrogenase, formate dehydrogenase, and D-amino acid dehydrogenase. Methods for measuring the activity of each of these enzymes are known to those skilled in the art and used routinely (see Iuchi and Lin, Proc. Natl. Acad. Sci. USA, 1888, 85:1888-1892, and the references cited therein).

Decreased ArcA activity in a modified bacterial cell compared to ArcA activity in a wild-type cell includes, but is not limited to, complete elimination of ArcA activity. Thus, a decrease in ArcA activity in a cell compared to ArcA activity in a wild-type cell includes, but is not limited to, complete elimination of ArcA activity. Complete elimination of ArcA activity encompasses a decrease of ArcA activity to such an insignificant level that it is undetectable using currently available detection methods.

In some aspects of the present invention, useful bacterial cells are modified to include a polypeptide that increases the conversion of NADH present in a cell to NAD (such as a polypeptide having NADH oxidase activity) and modified to include increased expression of enzymes involved in aerobic metabolism (for instance, modified to have decreased ArcA activity).

The modified bacterial cells described herein may also include other modifications, such as modifications which directly reduce carbon flow to acetate, and modifications which address the underlying metabolic and regulatory mechanisms which lead to acetate formation.

Modifications which directly reduce carbon flow to acetate include, for example, elimination of phosphotransacetylase (encoded by the pta coding region) and/or acetate kinase (Bauer et al., 1990, Appl. Environ. Microbiol., 56(5):1296-1302, Hahm et al., 1994, Appl. Microbiol Biotechnol., 42:100-107). Another example is the use of knockouts in poxB, ldhA, and pflB to result in reduced acetate formation (Lara et al., 2006, Biotechnol. Bioeng., 94(6):1164-1175). A related modification is to divert biochemicals residing at the end of glycolysis to compounds other than acetate. The acetolactate synthase coding region from Bacillus subtilis can be used to redirect pyruvate to acetoin (Aristidou et al., 1995, Biotechnol. Prog. 11:475-478). Similarly, a synthetic acetone operon from Clostridium acetobutylicum can divert some acetyl CoA to acetone (Bermejo et al., 1998, Appl. Environ. Microbiol. 64(3):1079-1085).

Modifications which address the underlying metabolic and regulatory mechanisms which lead to acetate formation include, for instance, the expression in the cell of pyruvate carboxylase (Gokarn et al,. 2001, Appl. Microbiol. Biotechnol. 5:188-195), or the expression in the cell of aspartase combined with supplying aspartate to the growth medium (Wang et al., 2006, J. Biotechnol. 124(2):403-411). Deleting coding sequences which regulate the TCA cycle can be used in the bacterial cells described herein to address the underlying causes of acetate formation. For example, a knockout in the fadR gene which represses the glyoxylate shunt can reduce acetate slightly, while the overexpression of PEP carboxylase in an fadR mutant can further reduce acetate (Farmer and Liao, 1997, Appl. Environ. Microbiol. 63(8):3205-3210).

Bacterial cells described herein and useful in the present invention include cells routinely used for the production of recombinant polypeptides for therapeutic, diagnostic, and industrial applications. Examples of bacterial cells that can be used include, for example, obligative aerobes (bacterial cells that require oxygen) and facultative aerobes (bacterial cells that do not require oxygen but can use it for respiration). Examples include members of the family Enterobacteriaceae, such as, for instance, genera including Escherichia, Salmonella, and Pseudomonas. Examples also include members of the family Bacillaceae, such as, for instance, genera including Bacillus. The invention is to be broadly understood as including methods of making the various embodiments of the bacterial cells useful in the invention described herein.

Methods for modifying bacterial cells can include the construction and introduction of polynucleotides into bacterial cells and the directed mutagenesis of coding regions present in bacterial cells. Such methods are known in the art (see, e.g., Sambrook et al, Molecular Cloning: A Laboratory Manual., Cold Spring Harbor Laboratory Press (1989), and Methods for General and Molecular Bacteriology, (eds. Gerhardt et al.) American Society for Microbiology, chapters 13-14 and 16-18 (1994)).

Bacterial cells useful in some aspects of the present invention can be made by transforming a host cell with a polynucleotide including a coding region encoding a suitable enzyme that increases the conversion of NADH to NAD, such as an enzyme having NADH oxidase activity. Since increased NADH oxidase activity in a bacterial cell has been observed to result in less acetate production, it is expected that any NADH oxidase will work in the present invention. Thus, the present invention is not limited by the NADH oxidase enzyme used. NADH oxidase enzymes have been identified in prokaryotes and eukaryotes, and include water-forming NADH oxidases and nonwater-forming NADH oxidases. The nucleotide sequences of many coding regions encoding NADH oxidases are known and readily available to the skilled person. Examples of prokaryotic NADH oxidases that have been characterized include water-forming NADH oxidase encoded by the nox gene from Streptococcus pneumoniae (Auzat et al., 1999, Mol. Microbiol., 34(5):1018-1028, GenBank Assession AF014458), the NADH oxidase encoded by S. mutans (Matsumoto et al., 1996, Biosci. Biotechnol. Biochem., 60(1):39-43, GenBank Assession D49951), the NADH oxidase encoded by the nox coding region from Serpulina hyodysenteriae (Stanton and Jensen, 1993, J. Bacteriol., 175(10):2980-2987, GenBank Assession U19610), and the NADH oxidase encoded by the nor coding region from Enterococcus faecalis (Ross and Claiborne, 1992, J. Mol. Biol., 227(3):658-671, GenBank Assession X68847). Also included are NADH oxidases that have been altered by trivial deletions, insertions, substitutions (such as conservative substitutions), or other alterations of an NADH oxidase such that the NADH oxidase activity remains.

In other aspects, a suitable enzyme may be one involved in aerobic metabolism. These enzymes have been identified in prokaryotes and eukaryotes, and the nucleotide sequences of coding regions encoding these enzymes are known and readily available to the skilled person. Examples of these enzymes include, for instance, those having the following activities: L-lactate dehydrogenase, D-amino acid dehydrogenase, acyl-CoA dehydrogenase, 3-hydroxyacetyl-CoA dehydrogenase, D-lactate dehydrogenase, pyruvate dehydrogenase, citrate synthase, aconitase, isocitrate dehydrogenase, 2-oxoglutarate dehydrogenase, succinate dehydrogenase, fumarase, malate dehydrogenase, and isocitrate lyase.

The polynucleotide encoding the suitable enzyme (for instance, an enzyme that increases the conversion of NADH to NAD or an enzyme involved in aerobic metabolism) can be inserted into a vector using routine techniques of molecular biology, and introduced into a bacterial cell by transformation. Methods for transformation of bacteria are well known in the art and used routinely by the skilled person, and include, for example, electroporation and chemical modification.

The vector can be circular or linear, single-stranded or double stranded, and can be DNA, RNA, or any modification or combination thereof. The vector can be a plasmid, a viral vector or a cosmid. Selection of a vector or plasmid backbone depends upon a variety of desired characteristics in the resulting construct, such as a marker sequence, plasmid reproduction rate, and the like. To facilitate replication inside a host cell, the vector preferably includes an origin of replication (known as an “ori”) or replicon. Suitable vectors are known in the art and used routinely.

The polynucleotide used to transform the host cell according to the invention can optionally include a promoter sequence operably linked to the nucleotide sequence encoding the enzyme to be expressed in the host cell. A promoter is a polynucleotide which causes transcription of genetic material. The invention is not limited by the use of any particular promoter, and a wide variety are known. Promoters act as regulatory signals that bind RNA polymerase in a cell to initiate transcription of a downstream (3′ direction) coding region. The promoter used in the invention can be a constitutive or an inducible promoter. It can be, but need not be, heterologous with respect to the host cell.

The polynucleotide used to transform the host cell can, optionally, include a Shine Dalgarno site (e.g., a ribosome binding site) and a start site (e.g., the codon ATG) to initiate translation of the transcribed message to produce the enzyme. It can, also optionally, include a termination sequence to end translation. A termination sequence is typically a codon for which there exists no corresponding aminoacetyl-tRNA, thus ending polypeptide synthesis. The polynucleotide used to transform the host cell can optionally further include a transcription termination sequence.

The polynucleotide used to transform the host cell optionally includes one or more marker sequences, which typically encode a polypeptide that inactivates or otherwise detects or is detected by a compound in the growth medium. For example, the inclusion of a marker sequence can render the transformed cell resistant to an antibiotic, or it can confer compound-specific metabolism on the transformed cell. Examples of a marker sequence are sequences that confer resistance to kanamycin, ampicillin, chloramphenicol and tetracycline.

A suitable enzyme can be expressed in the host cell from an expression vector containing a polynucleotide having the nucleotide sequence encoding the enzyme. Alternatively, the polynucleotide having the nucleotide sequence encoding the enzyme can be integrated into the host cell's chromosome. For instance, polynucleotides can be introduced into a bacterial chromosome using, for example, recombination.

Optionally, the bacterial cell may contain a heterologous coding region encoding a recombinant polypeptide. Bacterial cells are routinely used for the production of various types of recombinant polypeptides, and the present invention is not limited to the recombinant polypeptide expressed by the cell. Examples of recombinant polypeptides include, for instance, cytokines (including various hematopoietic factors and interleukins) interferons, growth factors, hormones, protease inhibitors, and antibiotics.

Bacterial cells useful in aspects of the present invention can be made by modifying an endogenous coding region involved in regulating aerobic metabolism. The coding regions that can be modified include arcA, arcB, fnr, soxR, soxS, oxyR, oxyS, crp, fur, and rpoS. The modification results in the removal of most activity of a polypeptide encoded by an arcA, arcB, fnr, soxR, soxS, oxyR, oxyS, crp, fur, or rpoS coding region, or complete elimination of the activity. For example, an arcA coding region can contain a mutation that results in the cell having decreased ArcA activity. Likewise, an arcB coding region can contain a mutation that results in the cell having decreased ArcA activity and decreased ArcB activity. Examples of mutations include the presence of one or more deletion, insertion, and/or substitution. A deletion may include deletion of part or an entire nucleotide sequence encoding ArcA, or deletion of a regulatory region of an arcA coding region. Typically, a mutation useful to produce a modified bacterial cell for use in one of the methods described herein is stable and non-reverting. A variety of methods that can be used to modify an arcA coding region in a bacterial cell are known and used routinely by the skilled person. For instance, DNA integration cassettes (also referred to as DNA mutagenic cassettes) can be used to replace a chromosomal arcA coding region in a wild-type cell by homologous recombination. Such cassettes typically include the mutation to be inserted, homologous nucleotide sequences to target the mutation to the arcA coding region, and a marker sequence.

A modified bacterial cell having decreased activity of a polypeptide encoded by an arcA, arcB, fnr, soxR, soxS, oxyR, oxyS, crp, fur, or rpoS coding region can contain a compound that impedes transcription of the coding region or translation of the mRNA. For instance, the cell can contain an antisense DNA or RNA, a double stranded RNA molecule, or a ribozyme that cleaves the arcA mRNA. The cell can contain an antibody or antibody-like molecules such as peptide aptamers that abolish the activity of an ArcA polypeptide.

The construction of mutations in a chromosomal copy of arcA, arcB, fnr, soxR, soxS, oxyR, oxyS, crp, fur, or rpoS, construction of an antisense DNA or RNA, or construction of a double stranded RNA molecule typically benefits from knowing the nucleotide sequence of the coding region in the cell to be modified. The nucleotide sequence of these coding regions in the bacterial cells useful in the present invention are known. For instance, an E. coli arcA coding region is the complement of nucleotides 4637613 to 4638329 of Genebank Accession No. U00096, and a Samonella typhimurium arcA coding region is the complement of nucleotides 4855370 to 4856086 of Genbank Accession NC_{—}003197. The actual nucleotide sequence of an arcA, arcB, fnr, soxR, soxS, oxyR, oxyS, crp, fur, or rpoS coding region in a bacterial cell may vary slightly from a publicly available sequence; however, the actual nucleotide sequence can be easily determined using routine methods to clone the coding region and determine the nucleotide sequence. Typically, the cloning of the coding region can be accomplished by use of the known nucleotide sequence in many routine techniques including, for instance, making primers for use in a polymerase chain reaction to amplify the coding region, or making a probe to screen a library for the coding region.

The present invention also includes methods for using the modified bacterial cells described herein. The bacterial cells of the present invention may be used to increase production desired products such as recombinant polypeptides, metabolites derived from glycolysis, the TCA cycle, or the combination thereof. The metabolites that are produced by the bacterial cells of the present invention are those that are or can be metabolically derived from glycolysis or the TCA cyle. Such metabolites include, but are not limited to, amino acids and organic acids such as pyruvic acid.

Typically, the bacterial cells have increased conversion of NADH present in a cell to NAD (such as increased NADH oxidase activity), increased expression of enzymes involved in aerobic metabolism (such as decreased ArcA activity), or the combination. When grown under certain conditions such cells have decreased acetate production relative to the wild-type cell (a bacterial cell identical to the modified cell except for the modification). Methods for determining the amount of acetate produced by a bacterial cell are known to the skilled person and are routine (see, e.g., Eiteman and Chastain, 1997, Anal. Chim. Acta, 338:69-70).

The methods may include providing a modified bacterial cell, culturing the cell, obtaining a product that is produced by the bacterial cell, and combinations thereof. The medium used to culture the bacterial cell and the volume of media used can vary. When a bacterial cell is being evaluated for the ability to produce a desired product, the bacterial cell can be grown in a suitable volume, for instance, 10 milliliters to 1 liter of medium. When a bacterial cell is being grown to obtain greater amounts of a desired product, the bacterial cell may be grown in a fermentor. Methods for growing bacterial cells in a fermentor are routine and known in the art. A bacterial cell is typically cultured in aerobic conditions. Typically, the cells are grown with sufficient oxygen so that they are not oxygen-limited.

Examples of useful growth media often used in smaller volumes are common commercially prepared media such as Luria Bertani broth, Sabouraud Dextrose broth or Yeast medium broth. Other defined or synthetic growth media may also be used and the appropriate medium for growth of a particular bacterial cell will be known by a person skilled in the art of microbiology or fermentation science.

Fermentation media useful in the present invention contains suitable carbon substrates, which includes but are not limited to monosaccharides such as glucose and fructose, oligosaccharides such as lactose or sucrose, polysaccharides such as starch or cellulose and unpurified mixtures from renewable feedstocks. Additionally the carbon substrate may also be one-carbon substrates such as carbon. Although it is contemplated that all of the above mentioned carbon substrates can be used in the present invention, glucose is typically present, and other carbon substrates may be added.

Fermentation may be batch, including fed-batch, or steady-state. A classical batch fermentation is a closed system where the composition of the media is set at the beginning of the fermentation and not subject to artificial alterations during the fermentation. A variation on a batch fermentation is a fed-batch fermentation. Fed-batch fermentation processes include a typical batch system with the exception that the substrate is added in increments as the fermentation progresses. Steady-state fermentation is an open system where a defined fermentation media is added continuously to a bioreactor and an equal amount of conditioned media is removed simultaneously for processing. Steady-state fermentation generally maintains the cultures at a constant high density where cells are primarily in log phase growth. It is contemplated that the present invention may be practiced using either batch, fed-batch or steady-state processes and that any known mode of fermentation would be suitable. Additionally, it is contemplated that cells may be immobilized on a substrate as whole cell catalysts and subjected to fermentation conditions.

The bacterial cells are typically grown under conditions that would cause the wild-type cells to produce acetate. Acetate production by bacterial cells of the present invention can produce less acetate than the wild-type bacterial cell grown under certain conditions. For example, the bacterial cells of the present invention can produce amounts of acetate that are at least 40%, at least 50%, at least 75%, or at least 100% less than the acetate produced by the wild-type bacterial cell grown under certain conditions. In some aspects, the amount of acetate produced by the bacterial cells of the present invention is completely eliminated. Complete elimination of acetate by a bacterial cell of the present invention encompasses the production of such an insignificant level of acetate that it is undetectable using currently available detection methods.

In those aspects of the present invention where fed-batch or steady-state conditions are used, oxygen and a carbon source (such as glucose) may be, and typically are, provided at rates that would cause the wild-type bacterial cell to produce acetate. The use of high carbon source feed rates with wild-type bacterial cells causes increased growth, which is desirable, but acetate is produced if the rate exceeds a threshold growth rate. The value of the threshold growth rate depends on the strain, but methods for determining the threshold growth rate are known to the skilled person and are routine. The use of bacterial cells modified as described herein permits higher carbon source feed rates without the acetate production observed with the wild-type bacterial cells. The higher carbon source feed rates results in a higher growth rate and increased production of recombinant polypeptide.

The desired products produced by the bacterial cells may be further isolated, and optionally purified, from the bacterial cells using protocols, methods and techniques that are well-known in the art. For instance, once polypeptides have been separated from cell debris, the recombinant polypeptide can be further purified using purification methods that are well known in the art. Suitable protein purification procedures can include fractionation on immunoaffinity or ion-exchange columns; ethanol precipitation; reverse phase HPLC; chromatography on silica or on an ion-exchange resin such as DEAE; chromatofocusing; SDS-PAGE; ammonium sulfate precipitation; gel filtration using, for example, Sephadex G-75; and ligand affinity chromatography. An “isolated” product, such as a polypeptide, means a polypeptide that is separate and discrete from the bacterial cell producing the product. A desired product may be purified, i.e., essentially free from any other polypeptide or polynucleotide and associated cellular products or other impurities. Recombinant polypeptides can be produced by bacterial cells of the present invention in amounts of at least 25%, at least 50%, at least 75%, or at least 100% greater than the recombinant polypeptide produced by the wild-type bacterial cell cultured under comparable conditions.

The present invention is illustrated by the following examples. It is to be understood that the particular examples, materials, amounts, and procedures are to be interpreted broadly in accordance with the scope and spirit of the invention as set forth herein.

### EXAMPLE 1

Overflow metabolism in the form of aerobic acetate excretion by Escherichia coli is an important physiological characteristic of this and other common industrial microorganisms. Although acetate formation occurs under conditions of high glucose consumption, the genetic mechanisms that trigger this phenomenon are not clearly understood. This Example describes the role of the NADH/NAD ratio (redox ratio) in overflow metabolism. The redox ratio in E. coli was modulated through the expression of a water-forming NADH oxidase. Using steady-state chemostat cultures, a strong correlation was demonstrated between acetate formation and this redox ratio. A genome-wide transcription analyses of a control E. coli strain and an E. coli strain overexpressing NADH oxidase was completed. The transcription results showed that in the control strain, several genes involved in the tricarboxylic acid (TCA) cycle and respiration were repressed as the glucose consumption rate increased. Moreover, the relative repression of these genes was alleviated by expression of NADH oxidase and the resulting reduced redox ratio. Analysis of a promoter binding site upstream of the genes which correlated with redox ratio revealed a degenerate sequence with strong homology with the binding site for ArcA. Deletion of arcA resulted in acetate reduction and increased the biomass yield due to the increased capacities of the TCA cycle and respiration. Acetate formation was completely eliminated by reducing the redox ratio through expression of NADH oxidase in the arcA mutant, even at a very high glucose consumption rate (Vemuri et al., Appl. Environ. Microbiol., 2006, 72(5):3653-3661).

Materials and Methods

Microorganisms and Media.

The E. coli K-12 strains MG1655 and QC2575 (MG1655 ΔarcA::Tet) were used in this study. QC2575 was obtained from D. Touati (l'Institut Jacques Monod, Paris, France). Growth and physiological characteristics were determined using defined media (Emmerling et al., 2002, J. Bacteriol. 184(1):152-164) composed of the following (per liter): 5 g glucose, 1.5 g NH₄Cl, 0.5 g NaCl, 7.8 g Na₂HPO₄.7H₂O, 3.5 g KH₂PO₄, 0.014 g CaCl₂.2H₂O, 0.246 g MgSO₄.7H₂O, 0.1 ml Antifoam C, 1 mg biotin, 1 mg thiamine, 100 mg ampicillin, and 10 ml trace metal solution. The trace metal solution contained the following (per liter): 16.68 g FeCl₃.6H₂O, 0.36 g ZnSO₄.7H₂O, 0.32 g CuSO₄.5H₂O, 0.2 g MnSO₄.H₂O, 0.18 g CoCl₂.6H₂O, 22.4 g EDTA, and 0.1 g NaMoO₄.2H₂O.

Construction of pTrc99A-nox.

The Streptococcus pneumoniae nox gene was amplified by PCR using pPANOX7 (M.-C. Trombe, U. Paul Sabatier, Toulouse, France) as template with Pfu DNA polymerase. Primers were designed based on the published S. pneumoniae nox gene sequence (Auzat et al., 1999, Mol. Microbiol. 34(5):1018-1028) and contained a BamHI restriction site and a Shine-Dalgarno sequence at the beginning of the amplified fragment and a PstI restriction site at the end of the amplified fragment (forward primer, 5′-TAC TAT GGA TCC AGG AGG TAA CAG CTA TGA GTA AAA TCG TTG TAG TCG GTG C-3′ (SEQ ID NO:1); reverse primer, 5′-ATA TAG TGA TCG ATA GCA GTC TGC AGT TAT TTT TCA GCC GTA AGG GCA GC-3′ (SEQ ID NO:2) [the underlined sequences are the BamHI, Shine-Dalgarno, ATG start, and PstI sites, respectively]). The resulting 1.4-kb PCR product was gel isolated, digested with BamHI and PstI, and ligated into the pTrc99A expression vector which had been digested with the same two restriction enzymes.

Chemostat Cultivation.

Carbon-limited chemostat cultures of 1.5-liter working volume were grown in 2.5-liter vessels (Bioflo II; New Brunswick Scientific, NJ) at 37° C., pH 7.0, and an agitation of 500 rpm. The airflow rate was maintained at 1.5 liter/min using mass flow controllers (Unit Instruments, Orange, Calif.) to ensure that the dissolved oxygen concentration remained above 40% of saturation at all growth rates studied. Measurements were made after the cells attained a steady state, which required at least 7 volume changes without any perturbation. The biomass formed was quantified by washing the cells with phosphate-buffered saline (pH 7.0) and drying for 12 h at 60° C. Glucose and organic acids in the feed and effluent were measured by high-performance liquid chromatography with a detection limit of about 0.05 g/liter (Eiteman et al., 1997, Anal. Chem. Acta. 338:69-70). Oxygen uptake rate and CO₂ evolution rate were calculated by measuring the effluent concentrations of oxygen and CO₂ (Ultramat 23 gas analyzer; Siemens, Germany). Each steady-state growth culture was freshly started from a single colony.

Quantification of NADH/NAD and Glycolytic Metabolites.

Metabolism was rapidly interrupted by extracting two 10-ml aliquots from a chemostat and plunging them into 40 ml methanol that had been prechilled for 4 h in a dry ice-ethanol bath. The cell pellets were resuspended in 0.2 M HCl (for extracting NAD) or 0.2 M NaOH (for extracting NADH), and the nucleotides were extracted by boiling the cell suspension. A cycling assay (Bernofsky et al., 1973, Anal. Biochem. 53(2):452-458) which involves the transfer of reducing equivalents from NADH ultimately to 3-(4,5-dimethylthiazol-2-yl)-2,5-diphenyltetrazolium bromide (MTT) was used to measure the specific nucleotides (Leonardo et al., 1993, J. Bacteriol. 175:870-878). The rate of reduction of MTT as measured at 570 nm was proportional to the concentration of NADH or NAD.

The intracellular concentrations of the key metabolites glucose-6-phosphate, fructose-6-phosphate, phosphoenolpyruvate (PEP), pyruvate, and acetyl-CoA were measured enzymatically by using cell extracts prepared by the perchloric acid method (Schaefer et al., 1999, Anal. Biochem. 270(1):88-96).

Global Transcription Profiling.

Changes in the expression of genes at various growth rates were identified using parallel two-color hybridization to whole-genome E. coli MG1655 spotted DNA arrays corresponding to 98.8% of the annotated open reading frames. The design, printing, and probing were previously described in detail (Khodursky et al., 2000, Proc. Natl. Acad. Sci. USA 97:12170-12175, Khodursky et al., 2003, Methods in Molecular Biology, ed. M. J. Brownstein, A. B. Khodursky, 224:61-78). After attaining a steady state at a predetermined dilution rate, samples were extracted from the chemostat and placed in RNAprotect buffer (QIAGEN, Valencia, Calif.), and the cell pellets were frozen at −80° C. Total RNA was extracted by the hot phenol-chloroform method and treated with DNase I in the presence of RNase inhibitor for subsequent labeling by reverse transcription with Cy3-dUTP and Cy5-dUTP fluorescent dyes. Total RNA from the strain containing pTrc99A plasmid was cultured at a dilution rate of 0.1 h⁻¹ and used as the common reference (always labeled with Cy3-dUTP) against which total RNA extracted from cells cultured at five higher equally spaced dilution rates (always labeled with Cy5-dUTP) was hybridized. Similarly, for the strain containing the pTrc99A-nox plasmid, total RNA extracted from cells cultured at a dilution rate of 0.06 h⁻¹ was the reference against which total RNA from cells cultured at five higher equally spaced dilution rates was hybridized. Differential gene expression between the two references (the NOX⁻ strain cultured at a dilution rate of 0.1 h⁻¹ and labeled with Cy3-dUTP, and the NOX⁺ strain cultured at a dilution rate of 0.06 h⁻¹ and labeled with Cy5-dUTP) was also measured to identify transcriptional changes due to the presence of the S. pneumoniae NADH oxidase. All the hybridizations were performed at least in triplicate by using biologically independent samples as described previously (Khodursky et al., 2003, Methods in Molecular Biology, ed. M. J. Brownstein, A. B. Khodursky, 224:61-78) and incubating the labeled mixture on the arrays at 65° C. overnight. The slide was subsequently washed and scanned using a GenePix 4000B microarray scanner (Axon Instruments, Union City, Calif.). The degree of labeling of the two dyes was quantified by measuring the intensity at a wavelength of 532 nm (for Cy3) or 635 nm (for Cy5). The relative expression of a gene was calculated as the base 2 logarithmic ratio of the background subtracted intensity from the Cy5 channel to the background subtracted intensity from the Cy3 channel, and the resulting value was referred to as the expression ratio.

Analysis of Gene Expression Data.

Our goal for the analysis of transcription data was to identify genetic changes that corresponded to physiological observations. Specifically, we were interested in identifying those genes whose expression was sensitive to a perturbation in the redox ratio (i.e., the NADH/NAD ratio). We calculated the Pearson correlation coefficient for the expression ratio of each gene with the redox ratio for NOX⁺ and NOX⁻ as a function of specific glucose consumption rate. Only those genes whose expression ratios had a high correlation coefficient (R>0.9 or R<−0.9) with the redox ratio were considered for further analysis. These highly correlated (or anticorrelated) genes were classified into 22 functional categories according to the method of Riley (Riley, 1998. Nucleic Acids Res. 26(1):54). Each functional category was tested for significant overrepresentation (P<0.05) by using a hypergeometric distribution (Jakt et al., 2001, Genome Res. 11(1):112-123). With a priori information on the distribution of the global gene set among the 22 categories, hypergeometric distribution measures the enrichment of a functional category based on the number of genes of that particular category appearing in the cluster. The P value for each category was calculated according to the following equation:

$$P = {\sum }_{x}^{N} \frac{( \begin{matrix}K \\ x\end{matrix} ) ( \begin{matrix}M - K \\ N - x\end{matrix} )}{( \begin{matrix}M \\ N\end{matrix} )}$$

where M is the total number of genes in the genome, x is the number of common genes, N is the total number of genes in the cluster, and K is the total number of genes in the functional category.

Only those genes from the significantly enriched functional categories were selected to study common regulatory mechanisms governing their expression. Any coregulation among these coexpressed genes was identified by searching for common transcription factor binding sites upstream of their transcription start sites. Sequences 300 bp upstream of the filtered genes were analyzed for common sequence motifs by using the hidden Markov model-based BioProspector software (Liu et al., 2001. Pac. Symp. Biocomput. 6:127-138).

Results

Physiological Response Due to NADH Oxidase Overexpression.

We used two isogenic strains that differ only in the presence of the nox gene, MG1655/pTrc99A (NOX⁻) and MG1655/pTrc99A-nox (NOX⁺). In batch cultures, NOX⁻ had a maximum growth rate (μₘₐₓ) of 0.70 h⁻¹ while NOX⁺ grew more slowly, with a μₘₐₓ of 0.51 h⁻¹. Based on these results, seven equally spaced dilution rates were selected for chemostat experiments to assess steady-state physiological and transcriptional responses to the overexpression of the nox gene. Both NOX⁻ and NOX⁺ exhibited fully respiratory metabolism until a critical dilution rate (or growth rate) was reached, above which respirofernentative metabolism was observed. The value of this critical dilution rate was about 0.4 h⁻¹ for the control, NOX⁻, and about 0.3 h⁻¹ for NOX⁺. No glucose was observed in the effluent for a dilution rate of less than 0.4 h⁻¹.

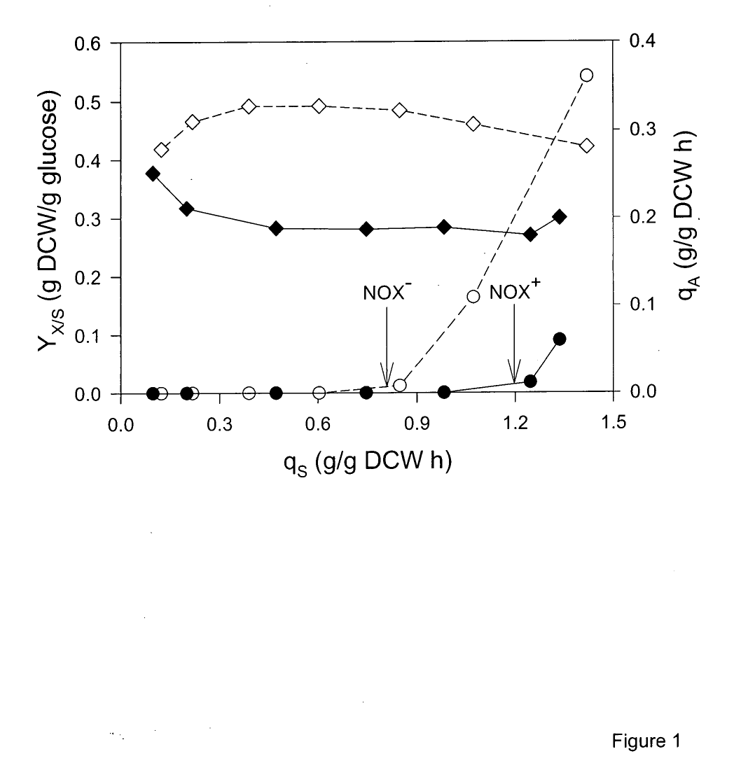

As shown in FIG. 1, acetate overflow is directly related to the rate at which the sole carbon source (glucose) is consumed, with acetate formation occurring only after glucose consumption surpasses some threshold rate. The presence of heterologous NADH oxidase had the effect of increasing the critical glucose consumption rate (q_{S}^{crit}) at which acetate first appeared and thereby delaying the entry of E. coli into respirofermentative overflow metabolism (FIG. 1). This transition between respiratory and respirofermentative metabolism occurred at a q_{S}^{crit} of 0.8 g/g dry cell weight (DCW) h for NOX⁻ and 1.2 g/g DCW h for NOX⁺. The expression of NADH oxidase therefore increased by 50% the value of q_{S}^{crit}. During respirofermentative metabolism, NOX⁺ exhibited a lower effluent acetate concentration and a lower specific acetate formation rate (q_{A}) than NOX⁻ at any given q_{S}. Biomass yield (Y_{X/S}) from glucose (g dry cell weight/g glucose consumed) was 0.42 to 0.48 g/g for NOX⁻ during respiratory metabolism but decreased during respirofermentative metabolism, consistent with a portion of the glucose carbon being diverted from biomass synthesis to acetate formation. For NOX⁺, Y_{X/S} remained 0.28 g/g at glucose consumption rates above 0.5 g/g DCW h (FIG. 1).

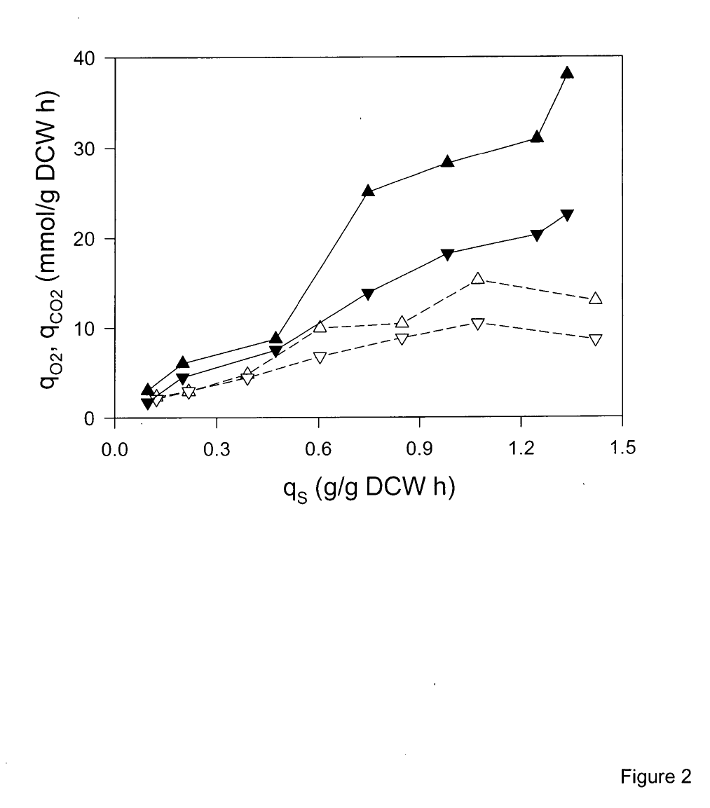

The specific oxygen consumption rate (q_{O2}) was twice as great for NOX⁺ as for NOX⁻ at any given value of q_{S} (FIG. 2), consistent with additional oxygen being required for increased oxidation of NADH to NAD. NOX⁺ also yielded a specific CO₂ evolution rate (q_{CO2}) that was about 50% greater than that of NOX⁻ for any q_{S} (FIG. 2), suggesting greater flux through CO₂-forming pathways (e.g., the TCA cycle) for NOX⁺. The results show that in the presence of NADH oxidase, cells diverted less carbon to biomass and acetate and more carbon to CO₂ at any given rate of glucose consumption. A carbon balance for NOX⁻ was within ±8% under all conditions, while for NOX⁺ the carbon balance was within ±15%, assuming identical biomass composition (and thus identical expression of biosynthetic genes). The redox balance closed for NOX⁻ within ±9%, while for NOX⁺ this balance was only within ±30%.

Intracellular Response Due to NADH Oxidase Overexpression.

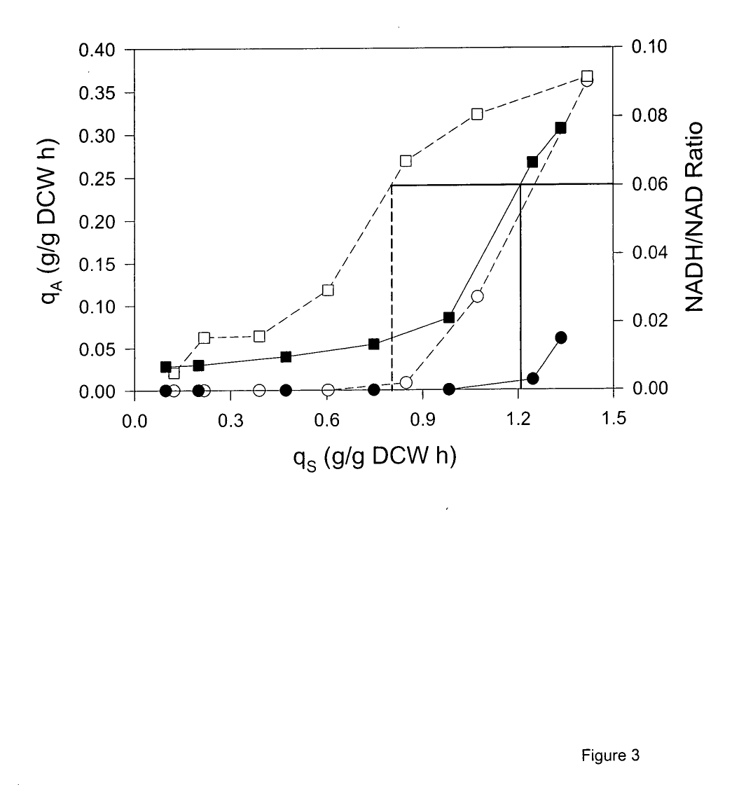

Since the expression of heterologous NADH oxidase in E. coli would be expected to influence the steady-state intracellular NADH and NAD concentrations, the concentrations of each cofactor were determined at each steady state for both strains. For both NOX⁻ and NOX⁺, the intracellular concentration of NAD changed less than 30%, while the NADH concentration changed more than 10-fold between the lowest and highest glucose consumption rates. Moreover, the NADH concentration increased more quickly for NOX⁻ at lower values of q_{S} than for NOX⁺. For example, at a q_{S} of about 0.10 g/g DCW h, the NADH concentration was 0.03 μmol/g DCW for both strains, while at a q_{S} of about 1.0 g/g DCW h, the NADH concentration was 0.53 μmol/g DCW for NOX⁻ but only 0.11 μmol/g DCW for NOX⁺. These changes are reflected in the NADH/NAD ratios (redox ratios) (FIG. 3). At any given value of q_{S}, the redox ratio was always greater for NOX⁻ than for NOX⁺. The redox ratio remained at 0.01 to 0.02 for both strains during respiratory metabolism but increased just prior to the onset of acetate overflow. Acetate formation for both strains occurred at an identical redox ratio of about 0.06 (FIG. 3). Clearly, this critical redox ratio marked a boundary between respiratory metabolism and respirofermentative metabolism. These results indicate a correlation between the redox ratio and acetate formation. What remains unclear is whether acetate formation is a consequence of the cells achieving the critical redox ratio, whether the increased redox ratio is caused by acetate formation, or whether these two phenomena are independent consequences of some underlying change in metabolism when the glucose consumption rate surpasses q_{S}^{crit}.

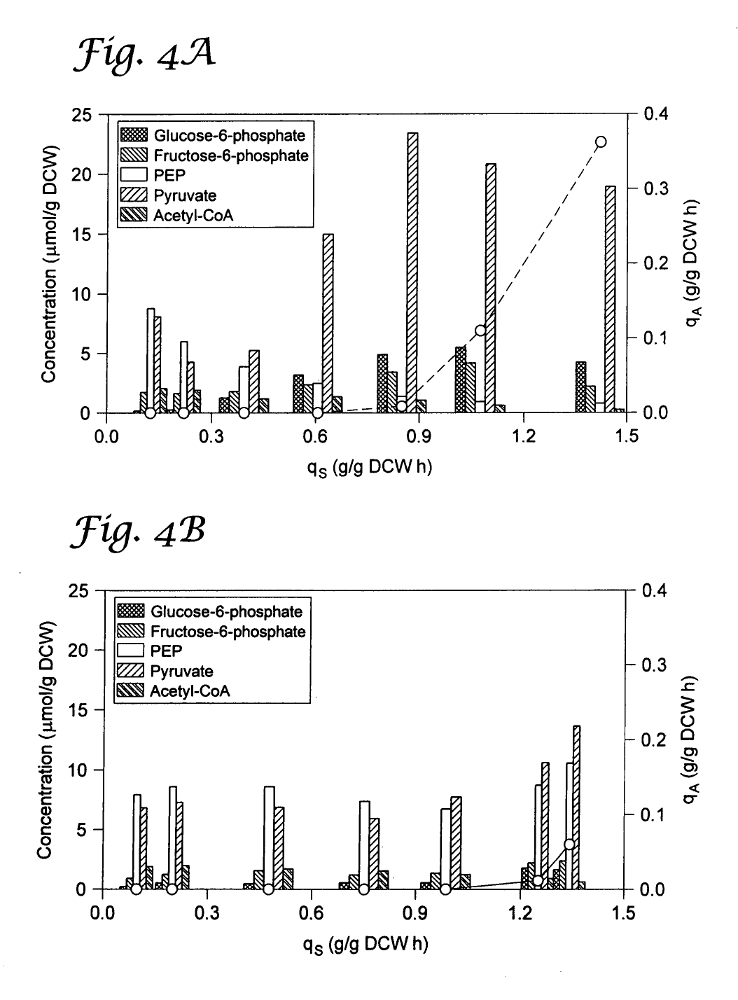

We measured the steady-state intracellular concentrations of key glycolytic intermediates in order to identify imbalances between glucose consumption and its subsequent metabolism that might occur. Steady-state pools of early glycolytic intennediates (glucose-6-phosphate and fructose-6-phosphate) increased with increasing q_{S} for NOX⁻ (FIG. 4A). Although pyruvate concentration increased, PEP concentration decreased markedly just at the onset of acetate formation, so that the pyruvate:PEP ratio increased from about 0.95 to 25. For NOX⁺, steady-state concentrations for each metabolite were essentially identical to those for NOX⁻ at the lowest q_{S}. However, the balance between PEP and pyruvate did not vary much with increasing q_{S} (FIG. 4B), with the pyruvate/PEP ratio increasing from about 0.90 to only 1.3.

Pyruvate and PEP in particular participate in a large number of biochemical reactions, and therefore, these metabolites tightly regulate a large portion of the metabolic network. The observed increase in the steady-state level of pyruvate could indicate increased fluxes in pathways using pyruvate as a substrate or as an enzyme activator, such as those pathways leading to the formation of acetate. Any shift in the pyruvate/PEP ratio suggests a shift in the degree of utilization of pathways that involve pyruvate compared to PEP. The correlation between acetate overflow and pyruvate/PEP ratio is consistent with an elevated intracellular level of pyruvate being a precursor to acetate formation.

Transcriptional Response to Increasing Glucose Consumption Rate.

Since most physiological events originate at the transcription level, we measured the transcriptional responses to changes in q_{S} for NOX⁻ and NOX⁺ strains to establish a genetic basis for the observed physiological changes. For each strain, we used a low value for q_{S} (corresponding to dilution rates of 0.1 h⁻¹ for NOX⁻ and 0.06 h⁻¹ for NOX⁺) as the reference. We first compared the transcription profiles for the two reference cultures (NOX⁻ grown at 0.1 h⁻¹ and NOX⁺ grown at 0.06 h⁻¹) to identify transcriptional changes only due to the presence of NADH oxidase. There were no significant transcriptional changes between these two reference cultures, suggesting limited influence of NADH oxidase at low q_{S}. This result is consistent with the similar physiological parameters (Y_{X/S}, q_{S}, q_{CO2}, and q_{O2}) and redox ratios observed for the two strains at low q_{S} values (FIGS. 1 to 3).

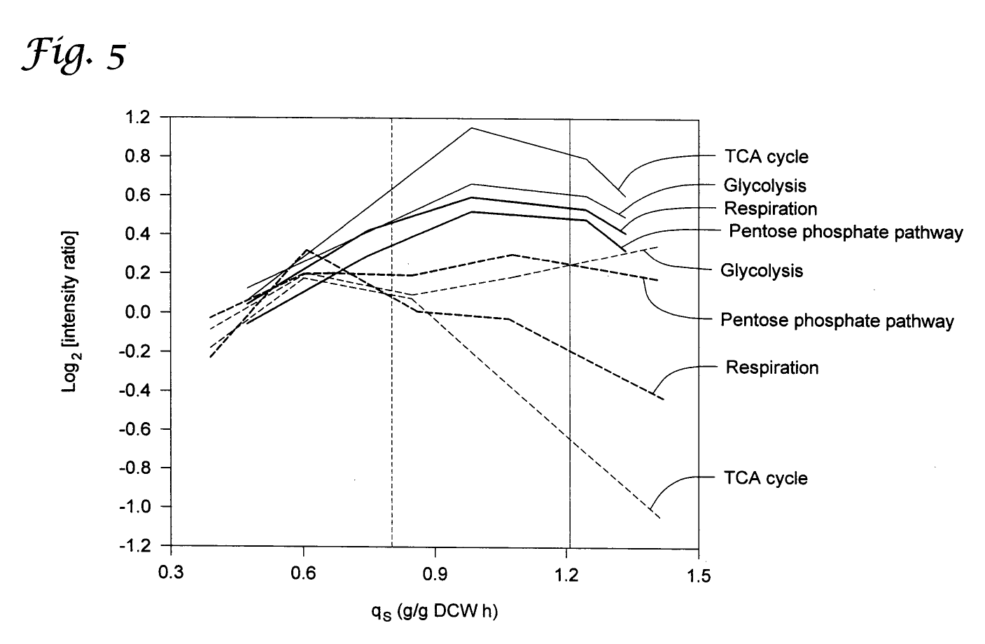

Next, we compared the transcriptional changes at higher values for q_{S} relative to the appropriate reference culture for each strain. In general, we did not observe drastic changes in gene expression, but many genes exhibited a reproducible monotonically increasing or decreasing behavior relative to the reference as q_{S} or growth rate increased. For NOX⁻, the expression of 427 genes varied significantly with q_{S} (P<0.01), while only 47 genes achieved this level of significance for NOX⁺ and only 21 genes were common to both subsets. Among the genes whose expression varied significantly for both NOX⁻ and NOX⁺ were key genes in the biosynthesis of threonine, serine, and nucleotides along with acs, which encodes acetyl-CoA synthetase. Since expression of these genes changed with q_{S} for both strains, their expression is presumably largely glucose consumption rate dependent and relatively insensitive to the redox state of the cell. The average expression ratios of all genes involved in the central metabolic pathways for NOX⁻ and NOX⁺ relative to their respective reference cultures are shown in FIG. 5 as functions of q_{S}. Transcription profiles of individual genes in these central metabolic pathways for the two strains are shown in FIG. 9.

The expression of most of the central metabolic genes (genes involved in the glycolysis TCA cycle, the pentose phosphate pathway, and respiration) increased during the respiratory phase of metabolism, but began to decrease just prior to respirofermentative metabolism for both NOX⁻ and NOX⁺, despite q_{S}^{crit} being 50% higher for NOX⁺. Regarding some of the key genes of interest to acetate formation, isocitrate dehydrogenase (icd) and citrate synthase (gltA) are inhibited by NADH and have been implicated in the control of flux in the TCA cycle (Holms, H. 1996, FEMS Microbiol. Rev. 19:85-116, Underwood et al., 2002, Appl. Environ. Microbiol. 68:1071-1081), and we also observed repression of these genes for NOX⁻ but induction for NOX⁺ as the glucose consumption rate increased. Thus, these genes appear to control TCA cycle flux at two levels: through enzyme activity and transcription. Interestingly, some TCA cycle genes (e.g., sucC, sucD) similarly appear to be controlled at both levels, while other TCA cycle genes (e.g., sucB, sdhC) encode enzymes not known to be controlled by the redox ratio but which show similar repression with increasing q_{S}. Induction of the acetate kinase gene (ackA) correlated with the formation of acetate for NOX⁻, while expression of the phosphotransacetylase gene (pta) was slightly repressed. For NOX⁺ the expression of ackA and pta increased with q_{S} during respiratory metabolism and remained constant during respirofermentative metabolism (FIG. 9). The key acetate consumption gene, acs, was severely (more than fivefold) repressed in both strains with increasing q_{S}. The pyruvate oxidase gene (poxB) was induced for NOX⁻ at low q_{S} values and repressed at high q_{S} values. Genes involved in aerobic respiration, such as the nuo operon (NADH dehydrogenase I chains), were generally repressed for NOX⁻ (FIG. 5; FIG. 9), and this repression was relieved for NOX⁺. The relative expression of the ndh gene encoding NADH dehydrogenase II, a primary source of NAD turnover under aerobic conditions, increased steadily with q_{S} for NOX⁻ and NOX⁺ during respiratory growth before saturation under respirofermentative conditions.

The relative expression of intermediate metabolic genes involved in the biosynthesis of amino acids and nucleotides increased with q_{S}, before either stabilizing or slightly decreasing at high q_{S} values for both NOX⁻ and NOX⁺ (FIG. 9). Moreover, we did not observe significant differences in the expression profiles of these genes between the strains at any given value of q_{S}, except for those involved in methionine and glycine biosynthesis. The relative expression of metABCEHJL genes either remained steady or decreased with q_{S} for NOX⁻, while these genes were significantly upregulated for NOX⁺. The glycine biosynthesis genes, particularly glyA, were repressed for NOX⁻, but the repression appeared to be reduced for NOX⁺. Purine and pyrimidine nucleotide biosynthesis genes monotonically increased with q_{S} for both strains. We also observed a repression in most of the transport genes at high q_{S} values for both NOX⁻ and NOX⁺. Among the genes encoding for symport or antiport proteins we found a few genes of the multifacilitator family that were upregulated (such as yhfC, yhaU, codB, uraA, and proP) while all others (including yjcG, lacY, gitS, gntT, dctA, tatC, melB, and nupG) were repressed (FIG. 9). Genes belonging to the ATP binding cassette transporters and the PEP-dependent phosphotransferase systems for the uptake of several sugars (including glucose) were also strongly repressed for both NOX⁻ and NOX⁺ as q_{S} increased (see FIG. 9). There was no particular trend observed in most unclassified genes except the gene encoding b4249 (a putative oxidoreductase), which was induced for NOX⁻ but was repressed for NOX⁺ as q_{S} increased.

Identifying Coregulation Among Co-Expressed Genes.

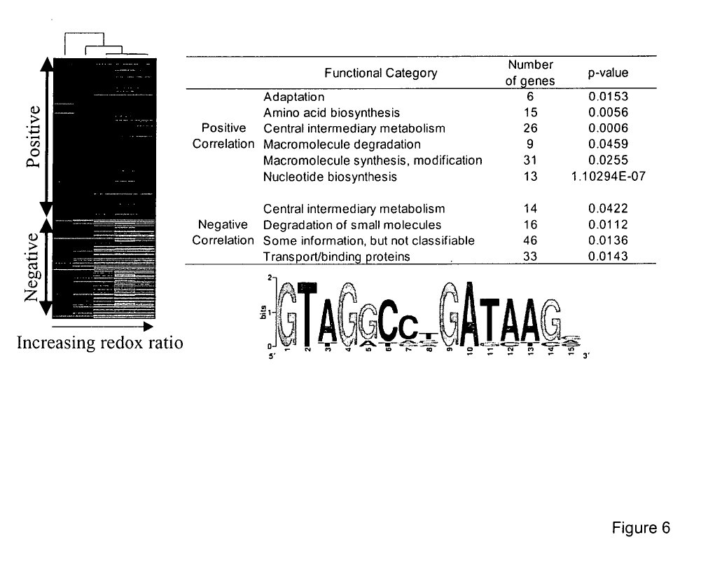

The approach governing our data analysis methodology was to first group coexpressed genes and then evaluate these gene groups for common regulatory mechanisms (see Materials and Methods). Genes involved in the biosynthesis of amino acids, cofactors, macromolecules, and nucleotides along with central and intermediary metabolic genes were positively correlated with q_{S} (R>0.9) for both NOX⁻ and NOX⁺. Among the genes that were negatively correlated with q_{S} (R<−0.9) were those responsible for the degradation of small molecules, transport proteins, and unclassified genes. Interestingly, we observed that only the genes involved in the biosynthesis of amino acids, cofactors, and nucleotides along with the central and intermediary metabolic genes were correlated (R>0.9) with the redox ratio for NOX⁻ (FIG. 6). There were also several partially classified genes in this subset, suggesting that the expression of a majority of these genes depends on the rate of glucose consumption and/or redox. While we identified strongly overrepresented sequences upstream of genes correlated with q_{S}, we could not relate these sequences with any of the known promoter binding sites. However, a significantly overrepresented (P<10⁻¹⁷⁰) sequence (FIG. 6) upstream of the genes correlated with the redox ratio for NOX⁻ was identified (by BioProspector) as the binding site for ArcA (Liu et al., 2004, J. Biol. Chem. 279(11):12588-12597, McGuire et al., 1999, Mol. Microbiol. 32:219-221, Pellicer et al., 1999, Mol. Gen. Genet. 261:170-176. The identification of an ArcA binding site upstream of genes that were correlated with the redox ratio (for NOX⁻) is consistent with a recent discovery that cellular redox state is the signal for the activation of ArcB signal transduction (Georgellis et al., 2001, Science 292(5525):2314-2316, Malpica et al., 2004, Proc. Natl. Acad. Sci. USA 101(36):13318-13323). Table 1 provides a complete list of genes in NOX⁻ that showed a reduction in expression by high NADH/NAD (negatively correlated with redox ratio) and which were detennined (by BIOPROSPECTOR) to have a binding site for ArcA. In light of these results, we speculated that the strong repression observed for several TCA cycle and respiratory genes in NOX⁻ at high q_{S} values might be relieved by deleting arcA.

Table 1. List of genes in E. coli MG1655 pTrc99A (NOX⁻) whose expression ratio was negatively correlated with NADH/NAD with increasing specific glucose consumption rate. Genes which were determined (by Bioprospector) to contain an ArcA binding site are also indicated (*).

| | Functional | Gene | |
|---|---|---|---|
| Genes | Category | Product | R |
| aceK* | Central | isocitrate dehydrogenase | — |
| | intermediary | kinase/phosphatase | 0.93 |
| | metabolism | | |
| acnB* | Energy | aconimtate hydrase B | — |
| | metabolism, | | 0.95 |
| | carbon | | |
| acs* | Fatty acid | acetyl-Co A synthetase | — |
| | biosynthesis | | 0.90 |
| add* | Central | adenosine deaminase | — |
| | intermediary | | 0.97 |
| | metabolism | | |
| adhP* | Energy | alcohol dehydrogenase | — |
| | metabolism, | | 0.97 |
| | carbon | | |
| aldA | Energy | aldehyde dehydrogenase, | — |
| | metabolism, | NAD-linked | 0.94 |
| | carbon | | |
| aldB | Degradation of | aldehyde dehydrogenase | — |
| | small molecules | B (lactaldehyde | 0.99 |
| | | dehydrogenase) | |
| allP* | Unknown | putative transport | — |
| | proteins, no | protein | 0.95 |
| | known homologs | | |
| araG* | Transport/binding | ATP-binding component | — |
| | proteins | of high-affinity L- | 0.92 |
| | | arabinose transport | |
| | | system | |
| araF* | Transport/binding | L-arabinose-binding | — |
| | proteins | periplasmic protein | 0.94 |
| argT* | Transport/binding | lysine-, arginine-, | — |
| | proteins | ornithine-binding | 0.97 |
| | | periplasmic protein | |
| aroM* | Amino acid | protein of aro operon, | — |
| | biosynthesis | regulated by aroR | 0.96 |
| b0024* | Unknown | orf, hypothetical | — |
| | proteins, no | protein | 0.99 |
| | known homologs | | |
| b1170* | Unknown | putative part of putative | — |
| | proteins, no | ATP-binding component | 0.90 |
| | known homologs | of a transport system | |
| b1394 | Degradation of | putative enzyme | — |
| | small molecules | | 0.91 |
| b1423 | Unknown | orf, hypothetical | — |
| | proteins, no | protein | 0.96 |
| | known homologs | | |
| b1440 | Transport/binding | putative transport | — |
| | proteins | protein | 0.90 |
| b1441* | Transport/binding | putative ATP-binding | — |
| | proteins | component of a | 0.97 |
| | | transport system | |
| b1443* | Transport/binding | putative transport system | — |
| | proteins | permease protein | 0.98 |
| b1444* | Some | putative aldehyde | — |
| | information but | dehydrogenase | 0.98 |
| | not calssifiable | | |
| b1486 | Transport/binding | putative transport system | — |
| | proteins | permease protein | 0.94 |
| b1488* | Unknown | orf, hypothetical protein | — |
| | proteins, no | | 0.93 |
| | known homologs | | |
| b1516 | Transport/binding | putative LACI-type | — |
| | proteins | transcriptional regulator | 0.99 |
| b1775 | Some | putative transport protein | — |
| | information, but | | 0.92 |
| | not classifiable | | |
| b1972 | Unknown | orf, hypothetical protein | — |
| | proteins, no | | 0.94 |
| | known homologs | | |
| b2080* | Unknown | orf, hypothetical protein | — |
| | proteins, no | | 1.00 |
| | known homologs | | |
| b2228 | Unknown | putative membrane | — |
| | proteins, no | protein | 0.90 |
| | known homologs | | |
| b2341 | Unknown | putative enzyme | — |
| | proteins, no | | 0.92 |
| | known homologs | | |
| b2390 | Unknown | orf, hypothetical protein | — |
| | proteins, no | | 0.96 |
| | known homologs | | |
| b2531 | Unknown | orf, hypothetical protein | — |
| | proteins, no | | 0.91 |
| | known homologs | | |
| b2659 | Unknown | orf, hypothetical protein | — |
| | proteins, no | | 0.99 |
| | known homologs | | |
| b2789 | Transport/binding | putative transport protein | — |
| | proteins | | 0.91 |
| b3001* | Transport/binding | putative reductase | — |
| | proteins | | 0.97 |
| b3045* | Laterally | IS2 hypothetical protein | — |
| | acquirred | | 0.91 |
| | elements | | |
| bax* | Some | putative ATP-binding | — |
| | information, but | protein | 0.94 |
| | not classifiable | | |
| bcsC* | Macromolecule | putative endoglucanase | — |
| | degradation | | 0.93 |
| bfd* | Adaptation | orf, hypothetical protein | — |
| | | | 0.93 |
| cdd | Central | cytidine/deoxycytidine | — |
| | intermediary | deaminase | 0.94 |
| | metabolism | | |
| cheA | Chemotaxis, | sensory transducer kinase | — |
| | motility | between chemo- signal | 0.95 |
| | | receptors and CheB and | |
| | | CheY | |
| cirA | Cell envelop | outer membrane receptor | — |
| | | for iron-regulated colicin | 0.93 |
| | | I receptor; porin; requires | |
| | | tonB gene product | |
| clpA* | Macromolecule | ATP-binding component | — |
| | degradation | of serine protease | 0.97 |
| creD | Global regulatory | tolerance to colicin E2 | — |
| | functions | | 0.94 |
| cspD* | Some | cold shock protein | — |
| | information, but | | 0.94 |
| | not classifiable | | |
| cycA | Transport/binding | transport of D-alanine, | — |
| | proteins | D-serine, and glycine | 0.95 |
| dcuC* | Transport/binding | transport of | — |
| | proteins | dicarboxylates | 0.93 |
| dgoT* | Transport/binding | D-galactonate transport | — |
| | proteins | | 0.95 |
| fadB* | Degradation of | 4-enzyme protein: 3- | — |
| | small molecules | hydroxyacyl-CoA | 0.99 |
| | | dehydrogenase; 3- | |
| | | hydroxybutyryl-CoA | |
| | | epimerase; delta(3)-cis- | |
| | | delta(2)-trans-enoyl-CoA | |
| | | isomerase; enoyl-CoA | |
| | | hydratase | |
| feaR* | Some | regulatory protein for 2- | — |
| | information, but | phenylethylamine | 0.91 |
| | not classifiable | catabolism | |
| fhuA | Cell envelop | outer membrane protein | — |
| | | receptor for ferrichrome, | 0.99 |
| | | colicin M, and phages | |
| | | T1, T5, and phi80 | |
| fruK* | Energy | fructose-1-phosphate | — |
| | metabolism, | kinase | 0.97 |
| | carbon | | |
| fruL | Energy | fruR leader peptide | — |
| | metabolism, | | 0.94 |
| | carbon | | |
| fucA | Degradation of | L-fuculose-1-phosphate | — |
| | small molecules | aldolase | 0.91 |
| fucO* | Degradation of | L-1,2-propanediol | — |
| | small molecules | oxidoreductase | 0.96 |
| fucR | Degradation of | positive regulator of the | — |
| | small molecules | fuc operon | 0.92 |
| fumC* | Energy | fumarase C = fumarate | — |
| | metabolism, | hydratase Class II; | 0.92 |
| | carbon | isozyme | |
| galS* | Degradation of | mgl repressor, galactose | — |
| | small molecules | operon inducer | 0.91 |
| gapC_2 | Energy | glyceraldehyde-3- | — |
| | metabolism, | phosphate dehydrogenase | 0.94 |
| | carbon | (second fragment) | |
| gatY | Degradation of | tagatose-bisphosphate | — |
| | small molecules | aldolase 1 | 0.93 |
| glpD* | Energy | sn-glycerol-3-phosphate | — |
| | metabolism, | dehydrogenase (aerobic) | 0.90 |
| | carbon | | |
| glpF* | Transport/binding | facilitated diffusion of | — |
| | proteins | glycerol | 0.98 |
| glpK | Central | glycerol kinase | — |
| | intermediary | | 0.99 |
| | metabolism | | |
| glpQ | Central | glycerophosphodiester | — |
| | intermediary | phosphodiesterase, | 0.92 |
| | metabolism | periplasmic | |
| glpT | Transport/binding | sn-glycerol-3-phosphate | — |
| | proteins | permease | 0.90 |
| gntP | Transport/binding | gluconate transport | — |
| | proteins | system permease 3 | 0.94 |
| hcaB* | Degradation of | 2,3-dihydroxy-2,3- | — |
| | small molecules | dihydrophenylpropionate | 0.97 |
| | | dehydrogenase | |
| hcaR | Degradation of | transcriptional activator | — |
| | small molecules | of hca cluster | 0.92 |
| hisQ* | Transport/binding | histidine transport system | — |
| | proteins | permease protein | 0.90 |
| hyfD | Energy | hydrogenase 4 membrane | — |
| | metabolism, | subunit | 0.98 |
| | carbon | | |
| hyfR | Some | putative 2-component | — |
| | information, but | regulator, interaction | 0.94 |
| | not classifiable | with sigma 54 | |
| idnD | Degradation of | L-idonate dehydrogenase | — |
| | small molecules | | 0.90 |
| idnO | Degradation of | 5-keto-D-gluconate 5- | — |
| | small molecules | reductase | 0.99 |
| kdpD* | Global regulatory | sensor for high-affinity | — |
| | functions | potassium transport | 0.91 |
| | | system | |
| lacA | Degradation of | thiogalactoside | — |
| | small molecules | acetyltransferase | 0.93 |
| lycV | Laterally | bacteriophage lambda | — |
| | acquirred | lysozyme homolog | 0.90 |
| | elements | | |
| melB* | Transport/binding | melibiose pennease II | — |
| | proteins | | 0.91 |
| mhpR | Some | transcriptional regulator | — |
| | information, but | for mhp operon | 0.95 |
| | not classifiable | | |
| mopA* | Cell diision | GroEL, chaperone | — |
| | | hsp60, peptide- | 0.91 |
| | | dependent ATPase, | |
| | | heat shock protein | |
| mtlD | Degradation of | mannitol-1-phosphate | — |
| | small molecules | dehydrogenase | 0.95 |
| narK* | Transport/binding | nitrite extrusion protein | — |
| | proteins | | 0.93 |
| nrdH | Biosynthesis of | glutaredoxin-like protein; | — |
| | cofactors, carriers | hydrogen donor | 0.98 |
| nrdl | Central | off, hypothetical protein | — |
| | intermediary | | 0.96 |
| | metabolism | | |
| paaB* | Degradation of | orf, hypothetical protein | — |
| | small molecules | | 0.92 |
| phnF | Some | putative transcriptional | — |
| | information, but | regulator | .099 |
| | not classifiable | | |
| ppsA | Central | phosphoenolpyruvate | — |
| | intermediary | synthase | 0.91 |
| | metabolism | | |
| prpB* | Some | putative | — |
| | information, but | phosphonomutase 2 | 0.91 |
| | not classifiable | | |
| prpC | Some | putative citrate synthase; | — |
| | information, but | propionate metabolism | 0.96 |
| | not classifiable | | |
| prpD | Degradation of | orf, hypothetical protein | — |
| | small molecules | | 0.95 |
| pqqL* | Biosynthesis of | putative peptidase | — |
| | cofactors, carriers | | 0.94 |
| rbsD* | Transport/building | D-ribose high-affinity | — |
| | proteins | transport system; | 0.95 |
| | | membrane-associated | |
| | | protein | |
| recN | Macromolecule | protein used in | — |
| | synthesis, | recombination and DNA | 0.95 |
| | modification | repair | |
| rhaR* | Degradation of | positive regulator for | — |
| | small molecules | rhaRS operon | 0.96 |
| rmf | Ribosome | ribosome modulation | — |
| | constituents | factor | 0.90 |
| rpoS* | global regulatory | RNA polymerase, sigma | — |
| | functions | S (sigma38) factor; | 0.93 |
| | | synthesis of many growth | |
| | | phase related proteins | |
| rspA | Global regulatory | starvation sensing protein | — |
| | functions | | 0.98 |
| sodA | Protection | superoxide dismutase, | — |
| | responses | manganese | 0.90 |
| spr* | Some | putative lipoprotein | — |
| | information, but | | 0.93 |
| | not classifiable | | |
| thiD* | Biosynthesis of | phosphomethyl- | — |
| | cofactors, carriers | pyrimidine | 0.92 |
| | kinase | | |
| thrS | Macromolecule | threonine tRNA | — |
| | synthesis, | synthetase | 0.91 |
| | modification | | |
| tra5_2 | Some | IS3 putative transposase | — |
| | information, but | | 0.97 |
| | not classifiable | | |
| tra5_3 | Some | IS3 putative transposase | — |
| | information, but | | 0.91 |
| | not classifiable | | |
| trkH* | Transport/binding | potassium uptake, | — |
| | proteins | requires TrkE | 0.91 |
| ttdA* | Energy | L-tartrate dehydratase, | — |
| | metabolism, | subunit A | 0.90 |
| | carbon | | |
| ugpE | Transport/binding | sn-glycerol 3-phosphate | — |
| | proteins | transport system, integral | 0.96 |
| | | membrane protein | |
| ugpQ | Central | glycerophosphodiester | — |
| | intermediary | phosphodiesterase, | 0.98 |
| | metabolism | cytosolic | |
| wzxC* | Transport/binding | probable export protein | — |
| | proteins | | 0.94 |
| xylF | Transport/binding | xylose binding protein | — |
| | proteins | transport system | 0.91 |
| xylG | Transport/binding | putative ATP-binding | — |
| | proteins | protein of xylose | 0.98 |
| | | transport system | |
| xylH* | Transport/binding | putative xylose transport, | — |
| | proteins | membrane component | 0.95 |
| xylR* | Some | putative regulator of xyl | — |
| | information, but | operon | 0.90 |
| | not classifiable | | |
| yafH | Some | putative acyl-CoA | — |
| | information, but | dehydrogenase (EC | 0.93 |
| | not classifiable | 1.3.99.-) | |
| yafL | Unknown | putative lipoprotein | — |
| | proteins, no | | 0.98 |
| | known homologs | | |
| yaiC | Unknown | orf, hypothetical protein | — |
| | proteins, no | | 0.93 |
| | known homologs | | |
| yajO* | Unknown | putative NAD(P)H- | — |
| | proteins, no | dependent xylose | 0.98 |
| | known homologs | reductase | |
| ybeR* | Unknown | orf, hypothetical protein | — |
| | proteins, no | | 0.92 |
| | known homologs | | |
| ybgG | Some | putative sugar hydrolase | — |
| | information, but | | 0.91 |
| | not classifiable | | |
| ybhI | Transport/binding | putative membrane pump | — |
| | proteins | protein | 0.90 |
| ybiX* | Some | putative enzyme | — |
| | information, but | | 0.92 |
| | not classifiable | | |
| ychA | Unknown | orf, hypothetical protein | — |
| | proteins, no | | 0.92 |
| | known homologs | | |
| ycjC* | Unknown | orf, hypothetical protein | — |
| | proteins, no | | 0.90 |
| | known homologs | | |
| ycjT | Unknown | orf, hypothetical protein | — |
| | proteins, no | | 0.97 |
| | known homologs | | |
| ydbC* | Unknown | putative dehydrogenase | — |
| | proteins, no | | 0.94 |
| | known homologs | | |
| ydbD | Unknown | orf, hypothetical protein | — |
| | proteins, no | | 0.93 |
| | known homologs | | |
| ydcA | Unknown | orf, hypothetical protein | — |
| | proteins, no | | 0.92 |
| | known homologs | | |
| ydcF* | Unknown | orf, hypothetical protein | — |
| | proteins, no | | 0.92 |
| | known homologs | | |
| yddA | Transport building | putative ATP-binding | — |
| | proteins | component of a transport | 0.91 |
| | | system | |
| yeaL | Unknown | orf, hypothetical protein | — |
| | proteins, no | | 0.95 |
| | known homologs | | |
| yeaW | Some | orf, hypothetical protein | — |
| | information, but | | 0.94 |
| | not classifiable | | |
| yebG | Unknown | orf, hypothetical protein | — |
| | proteins, no | | 0.95 |
| | known homologs | | |
| yeeE* | Some | putative transport system | — |
| | information, but | permease protein | 0.94 |
| | not classifiable | | |
| yehZ | Some | putative transport system | — |
| | information, but | permease protein | 0.94 |
| | not classifiable | | |
| yeiC* | Some | putative kinase | — |
| | information, but | | 0.96 |
| | not classifiable | | |
| yfeH | Some | putative cytochrome | — |
| | information, but | oxidase | 0.97 |
| | not classifiable | | |
| yfhH* | Transport/binding | orf, hypothetical protein | — |
| | proteins | | 0.95 |
| ygbF | Unknown | orf, hypothetical protein | — |
| | proteins, no | | 0.94 |
| | known homologs | | |
| ygcE | Some | putative kinase | — |
| | information, but | | 0.93 |
| | not classifiable | | |
| ygfR* | Unknown | putative oxidoreductase | — |
| | proteins, no | | 0.92 |
| | known homologs | | |
| ygfT* | Some | putative oxidoreductase, | — |
| | information, but | Fe-S subunit | 0.90 |
| | not classifiable | | |
| yhfO* | Unknown | orf, hypothetical protein | — |
| | proteins, no | | 0.90 |
| | known homologs | | |
| yidK* | Some | putative cotransporter | — |
| | information, but | | 0.93 |
| | not classifiable | | |
| yieC* | Transport/binding | putative receptor protein | — |
| | proteins | | 0.94 |
| yigF* | Unknown | orf, hypothetical protein | — |
| | proteins, no | | 0.98 |
| | known homologs | | |
| yigN* | Some | putative alpha helix chain | — |
| | information, but | | 0.94 |
| | not classifiable | | |
| yihU* | Some | putative dehydrogenase | — |
| | information, but | | 0.92 |
| | not classifiable | | |
| yihV* | Some | putative kinase | — |
| | information, but | | 0.91 |
| | not classifiable | | |
| yjcG | Transport/binding | putative transport protein | — |
| | proteins | | 0.93 |
| yjcH* | Unknown | orf, hypothetical protein | — |
| | proteins, no | | 0.96 |
| | known homologs | | |
| yjhI | Some | putative regulator | — |
| | information, but | | 0.93 |
| | not classifiable | | |
| yjiI | Central | orf, hypothetical protein | — |
| | intermediary | | 0.94 |
| | metabolism | | |
| ylaC* | Unknown | orf, hypothetical protein | — |
| | proteins, no | | 0.91 |
| | known homologs | | |
| ymfN | Unknown | orf, hypothetical protein | — |
| | proteins, no | | 0.92 |
| | known homologs | | |
| yncB | Some | putative oxidoreductase | — |
| | information, but | | 0.96 |
| | not classifiable | | |
| ynjA | Unknown | orf, hypothetical protein | — |
| | proteins, no | | 0.91 |
| | known homologs | | |
| ytfj* | Unknown | orf, hypothetical protein | — |
| | proteins, no | | 0.94 |
| | known homologs | | |
| ytfQ | Transport/binding | putative LACI-type | — |
| | proteins | transcriptional regulator | 0.99 |

Characterization of arcA Mutant.

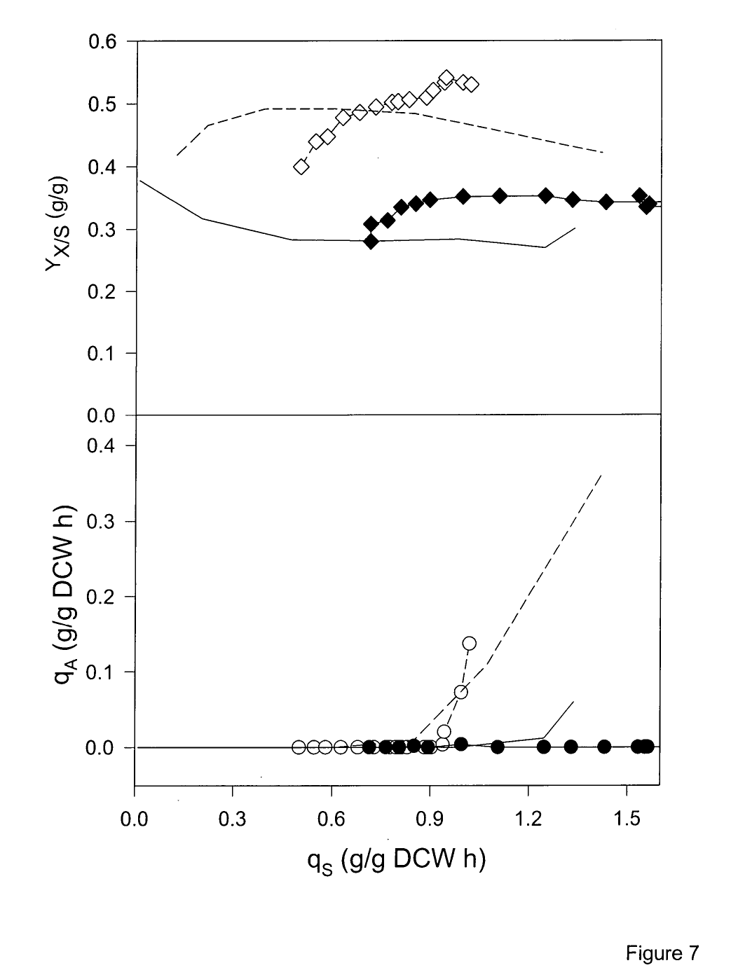

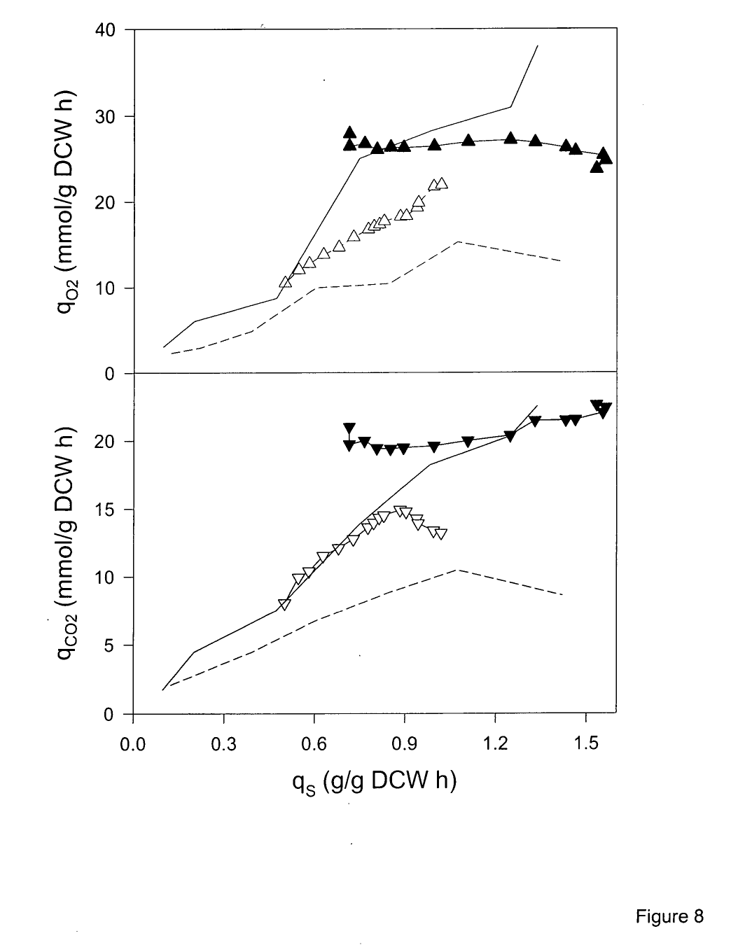

The identification of ArcA binding sites upstream of genes correlated with the redox ratio prompted us to characterize the phenotypes of QC2575/pTrc99A (ARCA⁻NOX⁻) and QC2575/pTrc99A-nox (ARCA⁻NOX⁺). In batch culture, the μₘₐₓ for ARCA⁻NOX⁻ (0.73 h⁻¹) was similar to that for NOX⁻, but the μₘₐₓ for ARCA⁻NOX⁺ (0.63 h⁻¹) was 20% greater than the value for NOX⁺. We performed accelerostat experiments (Kasemets et al., 2003, J. Microbiol. Methods, 55:187-200) for ARCA⁻NOX⁻ and ARCA⁻NOX⁺ to provide a pseudo-steady-state representation of physiological changes over a range of dilution rates (0.20 h⁻¹ to 0.54 h⁻¹). An accelerostat approximates the environment inside a chemostat, and these experiments began at steady state (after seven volume changes at a dilution rate of 0.2 h⁻¹). Once a steady state had been established, the dilution rate was slowly increased at a constant acceleration rate of 0.01 h⁻². For these experiments, the yield Y_{X/S} was about 30% lower for ARCA⁻NOX⁺ than for ARCA⁻NOX⁻ (FIG. 7). The most striking result of deleting arcA was the absence of acetate for ARCA⁻NOX⁺ even at the highest dilution rate studied. The value of q_{S}^{crit} for ARCA⁻NOX⁻ was 0.9 g/g DCW h, while we did not observe acetate even when q_{S} was equal to 1.5 g/g DCW h for ARCA⁻NOX⁺ (FIG. 7). The values of q_{O2} and q_{CO2} were 40% greater for ARCA⁻NOX⁻ than for NOX⁻, while they remained constant at about 27 mmol/g DCW h and 22 mmol/g DCW h, respectively, for ARCA⁻NOX⁺ (FIG. 8). Although parameters obtained with a steady-state chemostat may differ from those obtained with a pseudo-steady-state accelerostat, we have similarly observed no acetate formation for ARCA⁻NOX⁺ in batch fermentations.

Encouraged by these results, we measured pseudo-steady-state gene expression in ARCA⁻NOX⁻ relative to that in NOX⁻ when both strains were grown at a specific growth rate of 0.4 h⁻¹ (the critical growth rate for NOX⁻). The most important transcriptional changes in response to deletion of arcA occurred in genes involved in the TCA cycle and respiration. The expression of these genes increased over sevenfold for ARCA⁻NOX⁻ (see Table 2), presumably leading to the observed q_{CO2} and q_{O2} values being greater than those for NOX⁻. The fivefold increase in expression of the ptsG gene did not translate into a higher q_{S} value for ARCA⁻NOX⁻ than for NOX⁻, providing further evidence that glycolysis is not transcriptionally limited. Interestingly, the ptsG gene has also been demonstrated to be under ArcA control (Jeong et al., 2004, J. Biol. Chem. 279:38513-38518). Among the 110 genes showing significant difference between the strains (P<0.01), 30 of them are not classified while 21 are partly classified, including some regulatory genes (such as ispH [P=0.008]). Furthermore, we analyzed the 300 bp upstream of each gene whose expression of the ArcA binding box was statistically significant (P<0.01) in ARCA⁻NOX⁻ and comparedit to NOX⁻. BioProspector identified a binding site for ArcA upstream of about 60% of these genes. A comprehensive list of all genes with P values of <0.01 is given in Table 2.

TABLE 2

List of genes that are differentially expressed in response to deletion of arcA,
as determined by the p-value (only those genes with p<0.01 are shown). The
products of these genes and the functional category in which they are classified
are also shown. The expression ratios for each gene are the fold change in the
expression of that gene at a steady-state growth rate of 0.41 h⁻¹ in the arcA
strain (ARCA⁻NOX⁻) relative to that in control strain (NOX⁻) grown at the same
growth rate at steady-state. BioProspector was used to identify the presence of
a binding site for ArcA upstream of these genes. The binding site, if identified,
is also listed in the last column.

| | | | | | | |
|---|---|---|---|---|---|---|
| Gene | | | Fold | | Motif | |
| Name | Gene product | Functional Category | Ratio | p-value | Present | Binding Site |

| | | | | | | |
|---|---|---|---|---|---|---|
| agaB | PTS system, cytoplasmic, | Transport/binding proteins | 13.2 | 9.88E−09 | NO | |
| | N-acetylgalactosamine- | | | | | |
| | specific IIB component | | | | | |
| | 1 (EIIB-AGA) | | | | | |
| argY | Arginine tRNA synthetase | Ribosome constituent | 13.3 | 7.15E−09 | NO | |
| arsR | transcriptional repressor | Protection responses | 8.31 | 2.36E−03 | YES | tacacattcgttaagtca |
| | of chromosomal ars operon | | | | | SEQ ID NO. 3 |
| b0354 | nucleoprotein/polynucleo- | Some information, but | 9.3 | 3.64E−04 | YES | tcatttaacggtatgttg |
| | tide associated enzyme | not classifiable | | | | SEQ ID NO. 4 |
| b0513 | putative transport | Some information, but | 8.22 | 2.74E−03 | YES | caatttatccttaaacat |
| | | not classifiable | | | | SEQ ID NO. 5 |
| b0709 | putative transport protein | Transport/binding | 8.07 | 3.54E−03 | YES | aactttaaggaaacacaa |
| | | proteins | | | | SEQ ID NO. 6 |
| b0753 | putative homeobox protein | Some information, but | 7.55 | 8.03E−03 | YES | cctttgtttgttaattat |
| | | not classifiable | | | | SEQ ID NO. 7 |
| b1120 | putative nicotinic acid | Unknown proteins, no | 8.67 | 1.25E−03 | YES | tggcttatcgaatttccc |
| | mononucleotide:5,6- | known homologs | | | | SEQ ID NO. 8 |
| | dimethylbenzimidazole | | | | | |
| | (DMB) | | | | | |
| | phosphoribosyltransferase | | | | | |
| b1121 | homolog of virulence factor | Some information, but | 8.47 | 1.79E−03 | YES | cgtcggtttattaataag |
| | | not classifiable | | | | SEQ ID NO. 9 |
| b1148 | orf, hypothetical protein | Unknown proteins, no | 50.99 | 4.31E− | YES | aacagattcgttcagcaa |
| | | known homologs | | 165 | | SEQ ID NO. 10 |
| b1287 | putative oxidoreductase | Some information, but | 7.55 | 8.02E−03 | YES | gcaatgaacgaactcgaa |
| | | not classifiable | | | | SEQ ID NO. 11 |
| b1311 | putative binding-protein | Transport/binding | 11.42 | 2.25E−06 | YES | tgaactaccgaatcagct |
| | dependent transport protein | proteins | | | | SEQ ID NO. 12 |
| b1389 | orf, hypothetical protein | Degradation of small | 13.55 | 2.99E−09 | YES | gagtttaacgaagtaatt |
| | | molecules | | | | SEQ ID NO. 13 |
| b1527 | orf, hypothetical protein | Unknown proteins, no | 7.81 | 5.42E−03 | YES | ttggttaacaacctggct |
| | | known homologs | | | | SEQ ID NO. 14 |
| b1599 | possible chaperone | Unknown proteins, no | 7.71 | 6.32E−03 | YES | tagcgttttgttattcga |
| | | known homologs | | | | SEQ ID NO. 15 |
| b1631 | orf, hypothetical protein | Unknown proteins, no | 7.82 | 5.29E−03 | YES | ccggttaccgcttctacg |
| | | known homologs | | | | SEQ ID NO. 16 |
| b1671 | putative oxidoreductase, | Some information, but | 8.19 | 2.91E−03 | YES | gttgtacttgtttagcga |
| | Fe-S subunit | not classifiable | | | | SEQ ID NO. 17 |
| b1751 | orf, hypothetical protein | Unknown proteins, no | 14.12 | 4.01E−10 | YES | cccggtattgttatttat |
| | | known homologs | | | | SEQ ID NO. 18 |
| b1787 | orf, hypothetical protein | Unknown proteins, no | 10.01 | 7.77E−05 | YES | tcgattcgtaaaagtg |
| | | known homologs | | | | SEQ ID NO. 19 |
| b1871 | putative enzyme | Some information, but | 8.82 | 9.37E−04 | YES | tgaactgttgttcaacat |
| | | not classifiable | | | | SEQ ID NO. 20 |
| b1955 | orf, hypothetical protein | Unknown proteins, no | 9.25 | 3.98E−04 | YES | atgattaacgctacttat |
| | | known homologs | | | | SEQ ID NO. 21 |
| b2099 | orf, hypothetical protein | Unknown proteins, no | 8.04 | 3.72E−03 | YES | tcaggaaccggtaaacgg |
| | | known homologs | | | | SEQ ID NO. 22 |
| b2146 | putative oxidoreductase | Some information, but | 7.93 | 4.43E−03 | YES | ttttctatcgttaatatt |
| | | not classifiable | | | | SEQ ID NO. 23 |
| b2227 | orf, hypothetical protein | Unknown proteins, no | 12.45 | 1.09E−07 | YES | taaacataagttaactgg |
| | | known homologs | | | | SEQ ID NO. 24 |
| b2245 | orf, hypothetical protein | Some information, but | 8.84 | 9.03E−04 | YES | attggttttgtaaacctg |
| | | not classifiable | | | | SEQ ID NO. 25 |
| b2274 | orf, hypothetical protein | Unknown proteins, no | 7.67 | 6.67E−03 | YES | tcacttaaccattccatg |
| | | known homologs | | | | SEQ ID NO. 26 |
| b2386 | putative transport protein | Transport/binding | 10.55 | 2.14E−05 | YES | acgtttatgaaatcactt |
| | | proteins | | | | SEQ ID NO. 27 |
| b2433 | orf, hypothetical protein | Unknown proteins, no | 12.92 | 2.49E−08 | YES | aagatgaaccatgacgtc |
| | | known homologs | | | | SEQ ID NO. 28 |
| b2651 | orf, hypothetical protein | Unknown proteins, no | 8.05 | 3.69E−03 | YES | ttaataaacaaaaggtta |
| | | known homologs | | | | SEQ ID NO. 29 |
| b2670 | orf, hypothetical protein | Unknown proteins, no | 7.88 | 4.85E−03 | YES | ttaattaaaaataaatca |
| | | known homologs | | | | SEQ ID NO. 30 |
| b2756 | orf, hypothetical protein | Unknown proteins, no | 7.91 | 4.61E−03 | YES | ggttaacaccccatgc |
| | | known homologs | | | | SEQ ID NO. 31 |
| b2767 | orf, hypothetical protein | Unknown proteins, no | 8.11 | 3.32E−03 | YES | gtggcctgcgttaatgca |
| | | known homologs | | | | SEQ ID NO. 32 |
| b3865 | orf, hypothetical protein | Unknown proteins, no | 7.65 | 6.93E−03 | YES | gctttaaacaaacaatca |
| | | known homologs | | | | SEQ ID NO. 33 |
| b4256 | orf, hypothetical protein | Unknown proteins, no | 7.56 | 7.88E−03 | YES | gtattaaacaaatgtata |
| | | known homologs | | | | SEQ ID NO. 34 |
| b4405 | orf, conceptual translation | Macromolecule synthesis, | 10.75 | 1.31E−05 | YES | ttttttgccaaaacgcac |
| | in SwissProt is fused with | modification | | | | SEQ ID NO. 35 |
| | b4404 | | | | | |
| cyoC | cytochrome o ubiquinol | Energy metabolism, carbon | 9.81 | 1.21E−04 | YES | acctggatcgtgaaaagc |
| | oxidase subunit III | | | | | SEQ ID NO. 36 |
| dnaJ | chaperone with DnaK; heat | Folding and ushering | 11.15 | 4.64E−06 | NO | |
| | shock protein | proteins | | | | |
| eno | enolase | Energy metabolism, carbon | 7.58 | 7.68E−03 | YES | gcaggctttgtgaaagcc |
| | | | | | | SEQ ID NO. 37 |
| entE | 2,3-dihydroxybenzoate-AMP | Biosynthesis of cofactors, | 19.29 | 3.51E−20 | YES | caggttattgctgaactg |
| | ligase | carriers | | | | SEQ ID NO. 38 |
| fepE | ferric enterobactin | Transport/binding proteins | 10.95 | 7.90E−06 | YES | ggaatttacaaacttcag |
| | (enterochelin) transport | | | | | SEQ ID NO. 39 |
| focA | probable formate | Transport/binding proteins | 9.36 | 317E−04 | YES | aactcatttgttaatttt |
| | transporter (formate | | | | | SEQ ID NO. 40 |
| | channel 1) | | | | | |
| frdB | fumarate reductase, | Energy metabolism, carbon | 13.76 | 1.46E−09 | NO | |
| | anaerobic, iron-sulfur | | | | | |
| | protein subunit | | | | | |
| ftsL | cell division protein; | Cell division | 8.12 | 3.28E−03 | YES | gaccgtattgtgaaacgt |
| | ingrowth of wall at septum | | | | | SEQ ID NO. 41 |
| galK | galactokinase | Degradation of small | 15.3 | 4.34E−12 | YES | ttatggttggttatgaaa |
| | | molecules | | | | SEQ ID NO. 42 |
| gcd | glucose dehydrogenase | Degradation of small | 9.01 | 6.55E−04 | YES | gcggaatctgttaataaa |
| | | molecules | | | | SEQ ID NO. 43 |
| glcG | orf, hypothetical protein | Unknown proteins, no | 8.45 | 1.86E−03 | YES | tgcgttaacgcatcccga |
| | | known homologs | | | | SEQ ID NO. 44 |
| glpF | facilitated diffusion of | Transport/binding proteins | 7.93 | 4.44E−03 | YES | ctcgttaacgataagttt |
| | glycerol | | | | | SEQ ID NO. 45 |
| gltD | glutamate synthase, small | Central intermediary | 7.6 | 7.46E−03 | YES | ggtttcgcttacgtt |
| | subunit | metabolism | | | | SEQ ID NO. 46 |
| gusC | membrane-associated protein | Cell envelop | 11.93 | 5.18E−07 | NO | |
| hisQ | histidine transport system | Transport/binding proteins | 10.56 | 2.10E−05 | YES | gaaattagcgaaaaagta |
| | permease protein | | | | | SEQ ID NO. 47 |
| hupB | DNA-binding protein HU- | Macromolecule synthesis, | 17.35 | 5.78E−16 | YES | ttttgtctcgctaagtta |
| | beta, NS1 (HU-1) | modification | | | | SEQ ID NO. 48 |
| hybA | hydrogenase-2 small subunit | Energy metabolism, carbon | 7.7 | 6.44E−03 | YES | gatgttaacgctaaagag |
| | | | | | | SEQ ID NO. 49 |
| icdA | isocitrate dehydrogenase, | Energy metabolism, carbon | 7.84 | 5.15E−03 | YES | atcattaacaaaaaattg |
| | specific for NADP+ | | | | | SEQ ID NO. 50 |
| kdul | homolog of pectin degrading | Degradation of small | 7.67 | 6.64E−03 | YES | tgttttatttttaattga |
| | enzyme 5-keto 4-deoxy- | molecules | | | | SEQ ID NO. 51 |
| | uronate isomerase | | | | | |
| lytB | control of stringent | Global regulatory functions | 7.49 | 8.75E−03 | YES | gatttcaaccatccgctg |
| | response; involved in | | | | | SEQ ID NO. 52 |
| | penicillin tolerance | | | | | |
| mdaA | modulator of drug activity | Energy metabolism, carbon | 8.52 | 1.63E−03 | YES | cggtgttttgctcatgct |
| | A | | | | | SEQ ID NO. 53 |
| moeA | molybdopterin biosynthesis | Biosynthesis of cofactors, | 8.84 | 8.99E−04 | YES | agaatttttatgaattac |
| | | carriers | | | | SEQ ID NO. 54 |
| moeB | molybdopterin biosynthesis | Biosynthesis of cofactors, | 9.13 | 5.07E−04 | YES | agggttcacatatattta |
| | | carriers | | | | SEQ ID NO. 55 |
| motA | proton conductor component | Chemotaxis, motility | 8.3 | 2.42E−03 | YES | ctgtttaactgatacggt |
| | of motor; no effect on | | | | | SEQ ID NO. 56 |
| | switching | | | | | |
| nagC | transcriptional repressor | Central intermediary | 8.31 | 2.36E−03 | YES | aagaccatcgttaacggt |
| | of nag | metabolism | | | | SEQ ID NO. 57 |
| | (N-acetylglucosamine) | | | | | |
| | operon | | | | | |
| napF | ferredoxin-type protein: | Energy metabolism, carbon | 7.81 | 5.37E−03 | YES | tcttttagtgttaaattc |
| | electron transfer | | | | | SEQ ID NO. 58 |
| nmpC | outer membrane porin | Laterally acquirred | 17.48 | 3.12E−16 | YES | ctacttcacaaatcaaac |
| | protein; locus of qsr | elements | | | | SEQ ID NO. 59 |
| | prophage | | | | | |
| nuoB | NADH dehydrogenase I | Energy metabolism, carbon | 12.05 | 3.72E−07 | YES | aacagtatcgctaatcgt |
| | chain B | | | | | SEQ ID NO. 60 |
| nuoM | NADH dehydrogenase I | Energy metabolism, carbon | 7.78 | 5.62E−03 | YES | tgagaattcgttgaaaat |
| | chain M | | | | | SEQ ID NO. 61 |
| pdxH | pyridoxinephosphate oxidase | Biosynthesis of cofactors, | 10.36 | 3.38E−05 | YES | gtcagttttgtttacgat |
| | | carriers | | | | SEQ ID NO. 62 |
| phnA | orf, hypothetical protein | Unknown proteins, no | 8.12 | 3.29E−03 | YES | agcattaacattatctta |
| | | known homologs | | | | SEQ ID NO. 63 |
| phnI | phosphonate metabolism | Central intermediary | 8.07 | 3.52E−03 | NO | |
| | | metabolism | | | | |
| potD | spermidine/putrescine | Transport/binding proteins | 9.24 | 4.09E−04 | YES | gccgttaaaaatttattc |
| | periplasmic transport | | | | | SEQ ID NO. 64 |
| | protein | | | | | |
| potI | putrescine transport | Transport/binding proteins | 11.35 | 2.71E−06 | YES | gagtttttcaataaccgc |
| | protein; permease | | | | | SEQ ID NO. 65 |
| proK | proline tRNA synthetase | Ribosome constituent | 13.31 | 6.84E−09 | NO | |
| recC | DNA helicase, ATP- | Macrocolemule degradation | 13.32 | 6.55E−09 | YES | aacattaatgaacagtct |
| | dependent dsDNA/ssDNA | | | | | SEQ ID NO. 66 |
| | exonuclease V subunit, | | | | | |
| | ssDNA endonuclease | | | | | |
| rfaZ | lipopolysaceharide core | Macromolecule synthesis, | 7.87 | 4.94E−03 | YES | ggtattaaaaatgagatt |
| | biosynthesis | modification | | | | SEQ ID NO. 67 |
| rhaB | rhamnulokinase | Degradation of small | 8.54 | 1.58E−03 | YES | cagcaaattgtgaacatc |
| | | molecules | | | | SEQ ID NO. 68 |
| sdhB | succinate dehydrogenase, | Energy metabolism, carbon | 10.49 | 2.50E−05 | YES | cgccgaagcgtcaacatg |
| | iron sulfur protein | | | | | SEQ ID NO. 69 |
| sdhC | succinate dehydrogenase, | Energy metabolism, carbon | 8.16 | 3.08E−03 | YES | aatgattttgtgaacagc |
| | cytochrome b556 | | | | | SEQ ID NO. 70 |
| sodA | superoxide dismutase, | Protection responses | 7.55 | 8.00E−03 | YES | ttaattaactataatgaa |
| | manganese | | | | | SEQ ID NO. 71 |
| sseA | putative thiosulfate | Some information, but | 8.3 | 2.40E−03 | YES | gagagttttgctgaactc |
| | sulfurtransferase | not classifiable | | | | SEQ ID NO. 72 |
| sucD | succinyl-CoA synthetase, | Energy metabolism, carbon | 9.66 | 1.68E−04 | YES | gccgttctggttaacatc |
| | alpha subunit | | | | | SEQ ID NO. 73 |
| surA | survival protein | Some information, but | 12.12 | 3.03E−07 | YES | ttgtgatttgttgattta |
| | | not classifiable | | | | SEQ ID NO. 74 |
| tehB | tellurite resistance | Protection responses | 17.53 | 2.48E−16 | YES | atctttaccaattttatt |
| | | | | | | SEQ ID NO. 75 |
| tolR | putative inner membrane | Some information, but | 8.65 | 1.29E−03 | YES | cgcgtaaacaaactggaa |
| | protein involved in the | not classifiable | | | | SEQ ID NO. 76 |
| | tonB-independent uptake | | | | | |
| | of group A colicins | | | | | |
| valX | valine tRNA synthetase | Ribosome constituent | 8.2 | 2.86E−03 | NO | |
| yacH | putative membrane protein | Some information, but | 22.04 | 4.42E−27 | YES | ggctgaatcgttaaggat |
| | | not classifiable | | | | SEQ ID NO. 77 |
| yagI | putative regulator | Some information, but | 8.83 | 9.18E−04 | NO | |
| | | not classifiable | | | | |
| ybbF | orf, hypothetical protein | Unknown proteins, no | 7.51 | 8.55E−03 | YES | accgttagcgagtaat |
| | | known homologs | | | | SEQ ID NO. 78 |
| ybiI | orf, hypothetical protein | Unknown proteins, no | 8.54 | 1.56E−03 | YES | acacttaactgtacaagt |
| | | known homologs | | | | SEQ ID NO. 79 |
| ydjB | Nicotinamidase | Energy metabolism, carbon | 10.1 | 6.32E−05 | NO | |
| yhaA | putative kinase | Degradation of small | 8.23 | 2.71E−03 | NO | |
| | | molecules | | | | |
| yhaB | orf, hypothetical protein | Unknown proteins, no | 10.53 | 2.28E−05 | YES | cgtcattttgtgaatgca |
| | | known homologs | | | | SEQ ID NO. 80 |
| yhaE | putative dehydrogenase | Degradation of small | 17.16 | 1.39E−15 | YES | acctttaaaaaataacca |
| | | molecules | | | | SEQ ID NO. 81 |
| yhaF | orf, hypothetical protein | Degradation of small | 9.04 | 6.13E−04 | NO | |
| | | molecules | | | | |
| yhaV | orf, hypothetical protein | Unknown proteins, no | 8.3 | 2.42E−03 | YES | acattcaacaaggaaaga |
| | | known homologs | | | | SEQ ID NO. 82 |
| yhdV | orf, hypothetical protein | Unknown proteins, no | 8.47 | 1.77E−03 | YES | tatttttacaattcacat |
| | | known homologs | | | | SEQ ID NO. 83 |
| yhhI | putative receptor | Unknown proteins, no | 8.29 | 2.43E−03 | YES | attttgaacaatatggca |
| | | known homologs | | | | SEQ ID NO. 84 |
| yiaE | putative dehydrogenase | Some information, but | 8.1 | 3.38E−03 | YES | tcagttttccttcatcat |
| | | not classifiable | | | | SEQ ID NO. 85 |
| yiaN | putative membrane protein | Some information, but | 15.85 | 4.58E−13 | YES | ctggttacccattcctta |
| | | not classifiable | | | | SEQ ID NO. 86 |
| yicM | putative transport protein | Transport/binding proteins | 9.58 | 2.03E−04 | YES | ggattttacaaaaagctc |
| | | | | | | SEQ ID NO. 87 |
| yidJ | putative sulfatase | Unknown proteins, no | 8.77 | 1.03E−03 | YES | ggttttatcaaaccgcgc |
| | | known homologs | | | | SEQ ID NO. 88 |
| yidK | putative cotransporter | Some information, but | 15.4 | 2.93E−12 | YES | cacattttcgttaatcaa |
| | | not classifiable | | | | SEQ ID NO. 89 |
| yifE | orf, hypothetical protein | Unknown proteins, no | 7.91 | 4.59E−03 | YES | gtttttaacaattccgta |
| | | known homologs | | | | SEQ ID NO. 90 |
| yifK | putative amino acid/amine | Trasport/binding proteins | 9.92 | 9.62E−05 | YES | acccataacgataaccgg |
| | transport protein | | | | | SEQ ID NO. 91 |
| yihW | putative DEOR-type | Some information, but | 12.16 | 2.64E−07 | YES | atcttttttgtcactttt |
| | transcriptional regulator | not classifiable | | | | SEQ ID NO. 92 |
| yijC | orf, hypothetical protein | Unknown proteins, no | 8.08 | 3.51E−03 | YES | ttcattcacaatactgga |
| | | known homologs | | | | SEQ ID NO. 93 |
| yjjN | putative oxidoreductase | Some information, but | 10.3 | 3.96E−05 | NO | |
| | | not classifiable | | | | |
| ykfD | putative amino acid/amine | Transport/binding proteins | 7.86 | 5.02E−03 | YES | tctgttaacaaacgcggt |
| | transport protein | | | | | SEQ ID NO. 94 |
| yqgC | orf, hypothetical protein | Unknown proteins, no | 9.12 | 5.27E−04 | YES | acagttaacgactatcgc |
| | | known homologs | | | | SEQ ID NO. 95 |
| yqiB | putative enzyme | Some information, but | 7.4 | 9.98E−03 | YES | agcgttaaaaaatgagtg |
| | | not classifiable | | | | SEQ ID NO. 96 |
| yraM | putative glycosylase | Some information, but | 8.64 | 1.32E−03 | YES | ttgttgttcgttatggtc |
| | | not classifiable | | | | SEQ ID NO. 97 |

Discussion

The primary physiological consequences of providing additional means to oxidize excess NADH were reduction of acetate formation and biomass yield and a 50% increase in q_{S}^{crit}. An increase in q_{S}^{crit} at the expense of biomass formation indicates faster NADH turnover (i.e., both generation and consumption). Higher q_{O2} and q_{CO2} values for NOX⁺ also indicate higher glycolytic and TCA cycle flux. In the current study, increased NADH turnover due to overproduced NADH oxidase led to a 70% increase in glucose uptake at any given dilution rate (see FIG. S1 in the supplemental material), revealing a strong link between the rate of glycolysis and NAD availability. This result is in accordance with the view that control of glycolysis principally resides outside the pathway (Oliver, S. 2002. Nature 418(6893):33-34). A previous study (Jensen et al., 1992. J. Bacteriol. 174:7635-7641) with ATP synthase mutants similarly increased the rate of glycolysis. More recently, increasing ATP hydrolysis by overexpressing F₁-ATPase in E. coli was shown to increase the ADP pool and q_{S} values by 70% with a concomitant reduction in Y_{X/S} (Koebmann, et al., 2002, J. Bacteriol. 184(14):3909-3916), leading to the conclusion that demand for ATP could control glycolytic flux. Our experiments with overproducing NADH oxidase similarly increased q_{S} values and also reduced the intracellular redox ratio, and E. coli responded by upregulating genes involved in the TCA cycle and PDH complex, pathways that synthesize NADH and generate CO₂. These results provide experimental evidence to support the theory that glycolytic flux is controlled by the cellular demand for global cofactors such as NADH and ATP.

Although the rapid generation and subsequent oxidation of NADH in the NOX⁺ strain essentially introduces a futile NAD turnover, it reveals two very important metabolic events correlated with overflow metabolism in E. coli: both the redox ratio and the pyruvate/PEP ratio are correlated with the appearance of acetate. First, the redox ratio at the onset of acetate overflow was, surprisingly, identical for NOX⁻ and NOX⁺ (FIG. 3), indicating a relationship between the redox state of the cell and overflow metabolism. Since numerous reactions utilize or generate NADH, the redox ratio and overflow metabolism are likely to be the consequences of a complex network of metabolic events. The importance of NADH/NAD in by-product formation in E. coli has been previously demonstrated through increased reduction of NAD, which resulted not only in increased acetate but also in the appearance under aerobic conditions of typical fermentation products (Berrios-Rivera et al., 2002, Metabol. Eng. 4(3):217-229). In our study, TCA cycle genes which were generally repressed for NOX⁻ with increasing q_{S} values commonly showed less repression upon introduction of NADH oxidase. Considering that acetate overflow has thus far been assumed to be due to rate-limiting enzymes of the TCA cycle or the electron transport chain attaining maximum reaction velocity (Andersen et al., 1977, J. Biol. Chem. 252:4151-4156, El-Mansi et al., 1989, J. Gen. Microbiol. 135(11):2875-2883, Holms, H. 2001, Adv. Microb. Physiol. 45:271-340, Majewski et al., 1990, Biotechnol. Bioeng. 35:732-738), our results provide evidence that acetate overflow occurs as a consequence of transcriptional repression of the TCA cycle and respiratory genes (see FIG. S1 in the supplemental material). The introduction of NADH oxidase appears to delay the attainment of the critical redox ratio and limit acetate formation.

Also, the pyruvate/PEP ratio appears to be related to acetate formation (FIG. 4). As pyruvate is the branch point between respiration and fermentation and a precursor to several macromolecules, its level is highly regulated. In E. coli, PEP is a cosubstrate for glucose uptake and for the principal anaplerotic pathway during growth on glucose. Since acetate is produced from pyruvate directly (via pyruvate oxidase) or indirectly (via the PDH complex and acetate pathway), a 25-fold increase in the pyruvate/PEP ratio would shift the thermodynamic equilibrium towards pyruvate utilization by these pathways. Although an increase in the pyruvate/PEP ratio may not directly cause acetate overflow, the observed shift in the control strain NOX⁻ does signal the onset of a bottleneck at the entrance to the TCA cycle, which we have shown can be modulated by redox. The introduction of NADH oxidase served to decrease the pyruvate/PEP ratio and by mass action would make acetate formation less favorable. These results provide circumstantial evidence for considering pyruvate to be one ofthe candidate signaling metabolites for inducing the phosphorylation of ArcB (Iuchi et al., 1994, J. Bacteriol. 176(6):1695-1701). NADH was previously proposed as a possible signal (Iuchi et al., 1994, J. Bacteriol. 176(6):1695-1701), and more recent evidence (Georgellis et al., 2001, Science 292(5525):2314-2316) indicates that the cellularredox state is the signal for the activation of Arc regulation while pyruvate is an allosteric activator (Georgellis et al., 2001, Science 292(5525):2314-2316).

The strong correlation (R>0.9) between the redox ratio and the expression of genes involved in central and intermnediary metabolism and the biosynthesis of amino acids, cofactors, and nucleotides demonstrates the important regulatory control exerted by the redox state. The identification of binding sites for the ArcA protein upstream of many of these genes suggests redox-dependent regulation of the ArcAB system and is consistent with recent studies which propose that the redox state triggers the Arc system (Georgellis et al., 2001, Science 292(5525):2314-2316). Our analysis does not rule out the possibility of secondary regulation, and therefore the relationships between redox state, ArcA, and acetate overflow could be indirect. Although ArcA-mediated repression has been reported for many individual genes (operons), their integrated effect on induction of overflow metabolism has been largely overlooked. The reduced redox ratio for NOX⁺ may delay significant activation of the Arc system. For the ARCA⁻ strains, several of the TCA cycle and respiratory genes were induced and q_{CO2} was elevated, demonstrating greater TCA cycle flux. The resulting higher rate of NADH formation appears to be accommodated at least partly by the elevated q_{O2} which results from derepression of the respiratory chain in these strains. Importantly, although the q_{S}^{crit} was about 10% greater for ARCA⁻NOX⁻ compared to NOX⁻, acetate formation was even more pronounced for ARCA⁻NOX⁻ at higher levels of q_{S}. One possible explanation for this observation is that the heightened TCA cycle flux resulting from the absence of ArcA-mediated repression elevated NADH accumulation to a level beyond the capacity ofthe (derepressed) respiratory chain. Without a transcriptional mechanism to prevent further NADH formation in the TCA cycle, acetate formation may have occurred through some other mechanism (such as inhibition of citrate synthase). The overexpression of NADH oxidase in the arcA strain seems sufficient to provide another outlet for NADH oxidation and prevent acetate formation even at high glucose consumption rates.

In summary, these results using steady-state chemostats support a model in which an increase in the redox ratio contributes to a repression of the TCA cycle and to acetate formation, and they suggest that this overflow is due to transcriptional limitation. Providing another outlet for NADH turnover relieves TCA cycle gene repression and delays acetate formation. An arcA mutation delays the onset of acetate formation through the maintenance of both TCA cycle flux and respiration. Moreover, a strain with an arcA mutation and heightened NADH oxidase activity appears able to both maintain an elevated TCA cycle flux and alleviate NADH buildup, thereby preventing acetate formation altogether. Considering the deleterious impact of acetate on growth (Luli et al., 1990, Appl. Environ. Microbiol. 56:1004-1011) and recombinant protein production (Swartz, J. R. 2001, Curr. Opin. Biotechnol. 12:195-201) and the wide variety of genetic and process approaches proposed to reduce acetate formation, our findings provide evidence at the level of transcription for the cause of acetate overflow as well as offer a means to overcome it.

### EXAMPLE 2

Glycolytic flux is increased and acetate production is reduced in Escherichia coli by the expression of heterologous NADH-oxidase (NOX) from Streptococcus pnetinioniae coupled with the deletion of the arcA gene which encodes the ArcA regulatory protein. In this study, the overproduction of a model recombinant protein was examined in strains of E. coli expressing NOX with or without an arcA mutation. The presence of NOX or the absence of ArcA reduced acetate by about 50% and increased β-galactosidase production by 10-20%. The presence of NOX in the arcA strain eliminated acetate production entirely in batch fermentations and resulted in a 120% increase in β-galactosidase production (Vermuri et al., Biotechnol. Bioeng., 2006, 94(3):538-542, Eiteman and Altman, Trends Biotechnol., 2006, 24(11):530-536).

Materials and Methods

E. coli MG1655 was the host strain used in this study. QC2575 (MG1655 arcA::tet) was kindly provided by D. Touati (l'Institut Jacques Monod, Paris, France). β-galactosidase encoded by the lacZ gene (Fowler et al., 1978, J. Biol. Chem. 253(15):5521-5525) was expressed via the plasmid pACYC184-lacZ (March et al., 2002, Appl. Environ. Microbiol. 68(11):5620-5624), while water-forming NADH oxidase encoded by the nox gene from Streptococcus pneumoniae (Auzat et al., 1999, Mol. Microbiol. 34(5):1018-1028) was expressed via the plasmid pTrc99A-nox. The pTrc99A plasmid served as a control (Amann et al., 1988, Gene. 9:301-305). Each of the four strains, NOX⁻ (MG1655/pTrc99A), NOX⁺ (MG1655/pTrc99A-nox), ArcA⁻NOX⁻ (QC2575/pTrc99A) and ArcA⁻NOX⁺ (QC2575/pTrc99A-nox) were transfonned with pACYC184-lacZ and evaluated for β-galactosidase production.

The seed culture was started from a single colony and grown overnight at 37° C. in 10 mL of Luria-Bertani broth, 1 mL of which was transferred to 500 mL shake flasks containing 100 mL of the growth medium. The growth medium contained (per liter): 10 g glucose, 5 g NH₄Cl, 0.5 g NaCl, 10 g Na₂HPO₄.7H₂O, 5 g KH₂PO₄, 0.12 g MgSO₄.7H₂O, 0.15 g CaCl₂.2H₂O, 2.5 g LB, 1 mg biotin, 1 mg thiamine, and 10 mL of a trace metal solution which contained (per L): 16.67 g FeCl₃.6H₂O, 0.18 g ZnSO₄.7H₂O, 0.16 g CuSO₄.5H₂O, 0.21 g MnSO₄.H₂O, 0.18 g CoCl₂.6H₂O, 0.10 g Na₂MoO₄.2H₂O, 0.15 g Na₂B₄O₇.10H₂O, and 22.25 g Na₂EDTA.2H₂O. All cultures contained 100 mg/L ampicillin and 20 mg/L chloramphenicol to keep selective pressure on the pTrc99A and pACYC184 plasmids, respectively. The four strains were grown in batch cultures of 2.0 L working volume in 2.5 L benchtop fermenters (Bioflow III, New Brunswick Scientific, Co., Edison, N.J.) at 37° C. and with an air flowrate of 2 L/min. The pH was controlled at 7.0 with NH₄OH. The impeller stirring was initially 700 rpm and was automatically adjusted to ensure that the dissolved oxygen (DO) concentration always remained above 40% saturation. Protein production was induced after 1.5 hours of growth by adding IPTG to a final concentration of 1 mM. Culture samples were withdrawn from the fermenter and were stored at −20° C. until subsequent analyses.

Dry cell weight (DCW) of the culture was calculated from optical density measurements at 600 nm using the correlation: DCW=0.4788×OD₆₀₀, based on data from previous experiments. Residual glucose and acetate were analyzed by HPLC (Eiteman et al., 1997, Anal. Chem. Acta 338:69-70). CO₂ and O₂ in the off-gas were measured using a gas analyzer (ULTRAMAT 23, Siemens, Munich, Germany). Growth rate was determined by linear regression from a log plot of DCW versus time during the exponential growth phase.

Cell pellets from the samples extracted during the course of growth were resuspended in 50 mM phosphate buffer (pH 7.0) and were ruptured with a SLM-AMINCO FRENCH® Pressure Cell (Spectronic Instruments, Rochester, N.Y.). The cell extract was separated from the debris by centrifugation (4° C., 8000 rpm, 10 min). The activity of NADH oxidase in the cell extract was determined at 25° C., pH 7.0 and 340 nm by measuring the disappearance of 0.3 mM NADH in the presence of 0.3 mM EDTA (Lopez de Felipe et al., 1998, J. Bacteriol. 180(15):3804-3808). One unit (U) of NADH oxidase activity converts one μmole of NADH per minute to NAD. The activity of β-galactosidase was measured as described previously (Pardee et al., 1959, J. Mol. Biol 1:165-178), where one unit of activity produced 1 nmol of o-nitrophenol per min at 30° C. and pH 7. Total protein in the cell extracts was quantified using a BCA Protein Assay Kit (Pierce, Rockville, Ill.).

Results and Discussion

Overexpressing NADH oxidase (NOX) delays the formation of acetate in E. coli with increasing growth rate in chemostat cultures, and an arcA mutant alleviates the reduction of TCA cycle flux (see Example 1). Moreover, the overexpression of NOX in an arcA mutant eliminates acetate accumulation, even during rapid glucose consumption (see Example 1). Because three levels of acetate overflow were observed in continuous culture (high acetate in NOX⁻, moderate acetate in NOX⁺ and ArcA⁻NOX⁻, no acetate in ArcA⁻NOX⁺), we were interested in whether the expression of a model recombinant protein in these four strains correlated with acetate formation. Thus, in this current study these four E. coli strains having different respiratory capabilities were evaluated for their growth, acetate formation, respiratory parameters and recombinant β-galactosidase production during batch growth.

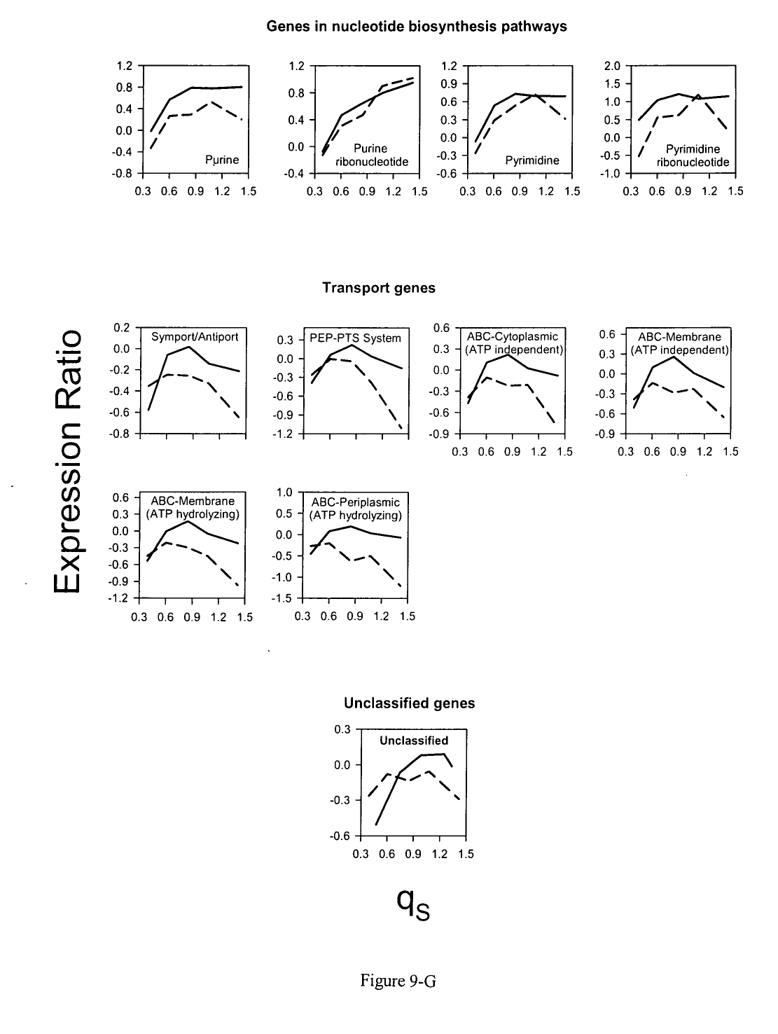

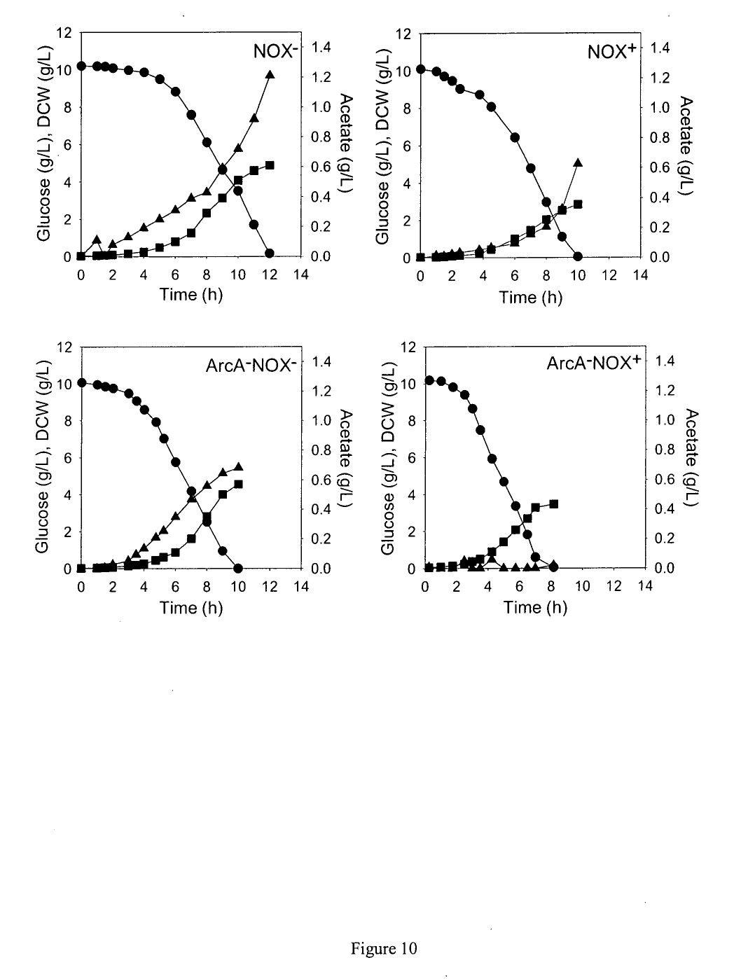

Compared with the control strain NOX⁻, the presence of heterologous NADH oxidase in NOX⁺ increased the maximum growth rate μₘₐₓ, but reduced the final biomass concentration and exponential phase yield, Y_{X/S} (Table 3, FIG. 10). The deletion of arcA did not affect the growth rate, and only decreased the exponential phase biomass yield slightly. The presence of NOX in the arcA strain, ArcA⁻NOX⁺, showed the highest value for μₘₐₓ of 0.71 h^{-1,} while the yield was indistinguishable from NOX⁺. Irrespective of the arcA deletion, the specific glucose consumption rate increased more than 100% in presence of nox for NOX⁺ and ArcA⁻NOX⁺ (Table 1). By supplying the cells with a means to generate more NAD at their maximum growth rate, glycolysis—a pathway requiring NAD as a cofactor—has been hastened. A conclusion consistent with this observation is that glycolysis (originally) is limited by the availability of NAD at the maximum specific growth rate. The final concentration of acetate was 1.2 g/L in NOX⁻, between 0.6 and 0.7 g/L for both NOX⁺ and ArcA⁻NOX⁻. We did not observe acetate accumulation in ArcA⁻NOX⁺ during batch growth (FIG. 10). Both strains with a functional ArcA (i.e., NOX⁻ and NOX⁺) showed exponential acetate accumulation as the stationary growth phase was approached. In contrast, ArcA⁻NOX⁻ showed a decrease in the rate of acetate accumulation in parallel with a decrease in cell growth rate as the stationary phase was entered. The rate of glucose uptake was faster as a result of the presence of NOX or the deletion of arcA while the combined effect ArcA⁻NOX⁺ resulted in the highest rate of glucose consumption (FIG. 10).

TABLE 1
Summary of growth and product formation in four strains of E. coli. The
values of specific growth rate (μₘₐₓ), biomass yield from glucose (Y_{X/S}) and
specific glucose consumption rate (qs) were calculated during the exponential
phase of growth, identified by the linear region in the ln(DCW) versus time plot.

| | | | qs | Final acetate | Final β-gal | NOX |
|---|---|---|---|---|---|---|
| Strain | μₘₐₓ (h⁻¹) | Y_{X/S} (g/g) | (g/g DCW h) | (g/L) | (kU/L) | (U/mg) |
| NOX⁻ | 0.55 | 0.48 | 0.27 | 1.21 | 26.8 | — |
| NOX⁺ | 0.66 | 0.29 | 0.60 | 0.63 | 30.0 | 0.50 |
| ArcA⁻ | 0.59 | 0.40 | 0.26 | 0.68 | 34.8 | — |
| NOX⁻ | | | | | | |
| ArcA⁻ | 0.71 | 0.30 | 0.61 | 0.02 | 59.5 | 0.31 |
| NOX⁺ | | | | | | |

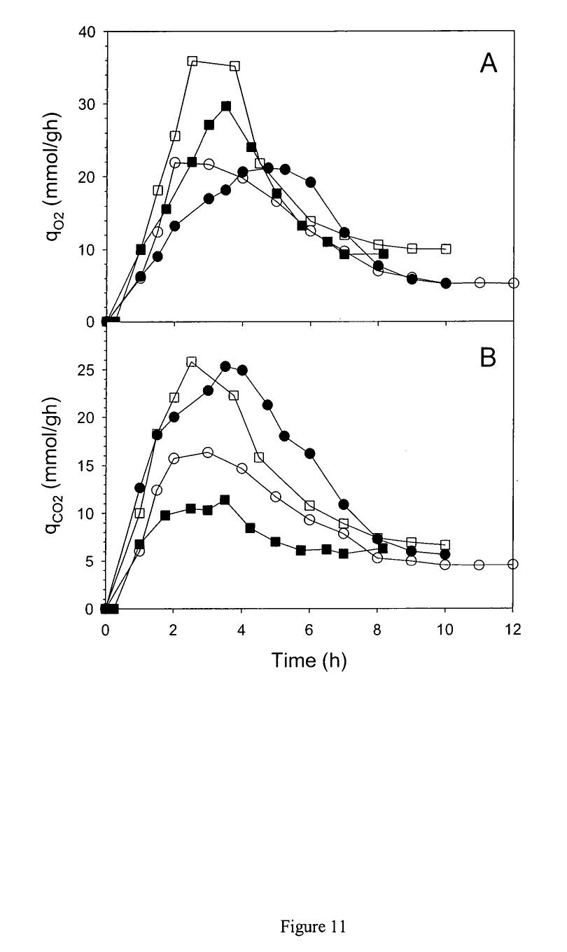

The specific oxygen uptake rate (q_{O2}) reached its maximum value during the mid-exponential phase of growth for all the strains and dropped rapidly as the cells progressed into early stationary phase (FIG. 11A). The maximum value of q_{O2} was 20% higher for NOX⁺ than for ArcA⁻NOX⁺ (FIG. 11A) while it remained at about 21 mmol/g DCW for both NOX⁻ and ArcA⁻NOX⁻. Strains containing NADH oxidase (NOX⁺ and ArcA⁻NOX⁺) consumed significantly higher oxygen compared to their isogenic control strains (NOX⁻ and ArcA⁻NOX⁻), a result consistent with greater NADH turnover in strains containing heterologous NOX. The specific CO₂ evolution rate (q_{CO2}) also achieved a maximum during the mid-exponential phase, but followed an interesting pattern in these strains. This maximum value of q_{CO2} was about 50% higher for NOX⁺ than for NOX⁻. However, an opposite trend was observed when the strain also contained the arca mutation. That is, the maximum value of q_{CO2} was 50% lower in ArcA⁻NOX⁺ compared with ArcA⁻NOX⁻ (FIG. 11B). The deletion of arcA increased q_{CO2} by about 50% compared to the control strain (NOX⁻). It is also interesting to note that the NOX⁺ and ArcA⁻NOX⁻ strains exhibited identical maximum values of q_{CO2} (about 25 mmol/gh) while accumulating similar final concentrations of acetate.

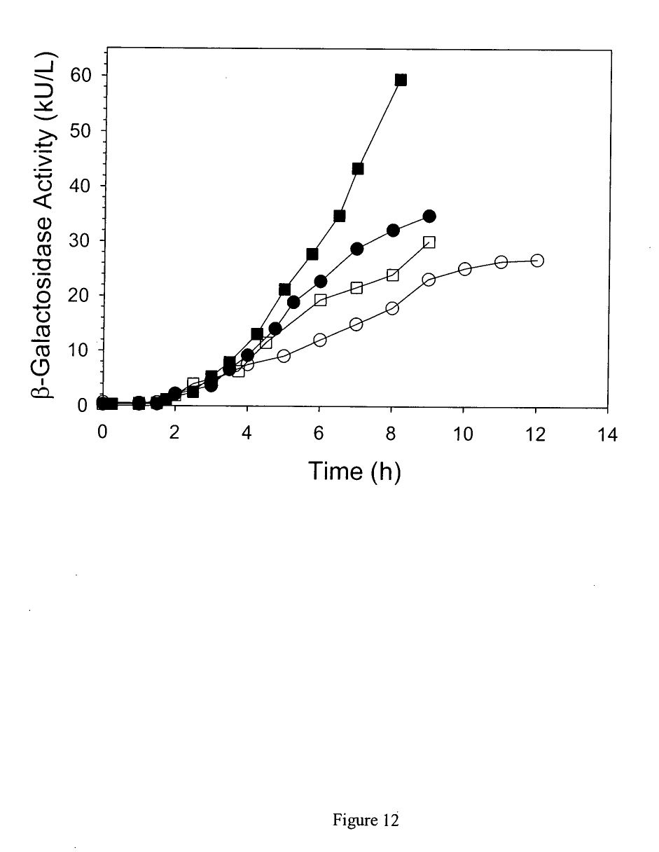

The reduction of fermentative/overflow behavior of E. coli during growth provided a very beneficial environment for the overproduction of recombinant protein (FIG. 12). The production of the model recombinant protein, β-galactosidase, was the lowest in NOX⁻. Both NOX⁺ and ArcA⁻NOX⁻ provided a modest 10-20% increase in β-galactosidase. However, the presence of NOX in an arcA mutant generated well over twice the amount of β-galactosidase than in the isogenic control. Since NOX⁻ accumulated the most acetate, followed by NOX⁺, ArcA⁻NOX⁻ and ArcA⁻NOX⁺, a strong (negative) correlation was observed between overflow metabolism and the production of recombinant proteins.

These results suggest that avoidance of respirofermentative metabolism is relevant for protein overproduction in E. coli. The NADH/NAD ratio and the intracellular pyruvate concentration are correlated with acetate overflow (see Example 1). Pyruvate is the precursor for acetate formed either through pyruvate oxidase or through phosphotransacetylase/acetate kinase formation and has been implicated to be a potential signaling molecule in the activation of the Arc system (Georgellis et al., 1999, Science 292(5525):2314-2316; Rodriguez et al., 2004, J. Bacteriol. 18697:2085-2090). Providing the cell with a means to reduce the NADH/NAD ratio while preventing the repression of the TCA cycle by the Arc system in ArcA⁻NOX⁺ provided additional carbon to the cell for biomass and hence for protein production. The presence of NOX or the deletion of arcA increased the CO₂ formation, consistent with a greater flux through the TCA cycle. It is not clear, however, why the ArcA⁻NOX⁺ strain generated the least CO₂. This might be explained by a particularly high anaplerotic flux in this strain, for example, through PEP carboxylase, which by supplying TCA cycle precursors could contribute to protein production.

The experiments conducted in this present study used batch conditions in which the maximum growth rate was achieved for each given strain. Many industrial fermentations are operated under fed-batch conditions in which the growth rate is limited by the rate at which a nutrient is supplied such as the carbon source glucose. Sufficient reduction of the growth rate serves as one means to prevent acetate formation by altogether avoiding overflow metabolism. Growth of ArcA⁻ and/or NOX⁺ strains in fed-batch mode under such low growth rates would likely provide no benefit to the cell for protein production, as the cells are not displaying overflow metabolism at that growth rate. The principal affect that ArcA⁻ or NOX⁺ would have in fed-batch operations is to increase the critical growth rate or glucose consumption rate at which acetate formation commences. Therefore, a higher nutrient feed rate and growth rate can be achieved without acetate formation.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set folth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A method comprising culturing a modified bacterial cell in aerobic conditions, wherein the modified bacterial cell comprises greater conversion of NADH to NAD than a wild-type bacterial cell or greater expression of an aerobic metabolism polypeptide than a wild-type bacterial cell, and wherein the modified bacterial cell produces less acetate during the culturing than the wild-type bacterial cell under comparable conditions.

2. The method of claim 1 wherein the modified bacterial cell comprises a heterologous coding region encoding a polypeptide having NADH oxidase activity.

3. The method of claim 1 wherein the modified bacterial cell comprises increased NADH oxidase activity.

4. The method of claim 1 wherein the modified bacterial cell comprises decreased ArcA activity.

5. The method of claim 4 wherein the modified bacterial cell comprises an endogenous arcA coding region or arcB coding region which comprises a mutation.

6. The method of claim 5 wherein the mutation comprises a deletion.

7. The method of claim 6 wherein the ArcA activity or ArcB activity is completely eliminated.

8. The method of claim 1 wherein the modified bacterial cell is an E. coli.

9. The method of claim 1 wherein the modified bacterial cell produces at least 40% less acetate than the wild-type bacterial cell under comparable conditions.

10. The method of claim 1 wherein the modified bacterial cell produces a recombinant polypeptide.

11. The method of claim 1 wherein the cell comprises greater conversion of NADH to NAD than a wild-type bacterial cell and greater expression of an aerobic metabolism polypeptide than a wild-type bacterial cell.

12. A method comprising culturing a modified bacterial cell in aerobic conditions, wherein the modified bacterial cell comprises increased NADH oxidase activity when compared to a wild-type bacterial cell, wherein the modified bacterial cell comprises decreased ArcA activity when compared to a wild-type cell, and wherein the modified bacterial cell produces less acetate during the culturing than the wild-type bacterial cell under comparable conditions.

13. The method of claim 12 wherein the modified bacterial cell comprises a heterologous coding region encoding a polypeptide having NADH oxidase activity.

14. The method of claim 12 wherein the modified bacterial cell comprises an endogenous arcA coding region or arcB coding region which comprises a mutation.

15. A method comprising culturing a modified bacterial cell in aerobic conditions and obtaining a desired product produced by the modified bacterial cell, wherein the modified bacterial cell comprises increased NADH oxidase activity when compared to a wild-type bacterial cell or decreased ArcA activity when compared to a wild-type bacterial cell, and wherein the modified bacterial cell produces more of the desired product than the wild-type bacterial cell under comparable conditions.

16. The method of claim 10 wherein the desired product is a metabolite or a recombinant polypeptide.

17. The method of claim 16 wherein the modified bacterial cell produces at least 25% more recombinant polypeptide than the wild-type bacterial cell.

18. The method of claim 15 further comprising isolating the desired product.

19. The method of claim 15 wherein the modified bacterial cell is an E. coli.

20. A method for increasing production of a recombinant polypeptide comprising expressing the recombinant polypeptide in a modified bacterial cell comprising greater conversion of NADH to NAD than a wild-type bacterial cell or greater expression of an aerobic metabolism polypeptide than a wild-type bacterial cell, wherein the amount of recombinant polypeptide produced in the modified bacterial cell is increased compared to the amount of recombinant polypeptide produced in the wild-type cell under comparable conditions.

21. The method of claim 20 wherein the modified bacterial cell comprises a heterologous coding region encoding a polypeptide having NADH oxidase activity.

22. The method of claim 21 wherein the modified bacterial cell comprises increased NADH oxidase activity.

23. The method of claim 20 wherein the modified bacterial cell comprises decreased ArcA activity.

24. The method of claim 23 wherein the modified bacterial cell comprises an endogenous arcA coding region or arcB coding region which comprises a mutation.

25. The method of claim 20 wherein the modified bacterial cell is an E. coli.

26. A modified bacterial cell which is an obligative aerobe or a facultative aerobe and comprises greater NADH oxidase activity than a wild-type bacterial cell and decreased ArcA activity when compared to the wild-type bacterial cell.

27. The modified bacterial cell of claim 26 wherein the modified bacterial cell comprises a heterologous NADH oxidase polypeptide.

28. The modified bacterial cell of claim 26 wherein the modified bacterial cell comprises an arcA coding region which comprises a mutation.

29. The modified bacterial cell of claim 28 wherein the modified bacterial cell is E. coli.
